(19) United States
(12) Patent Application Publication
(10) Pub. No.: US 2021/0187002 A1
(43) Pub. Date: Jun. 24, 2021

(54) ANTIMALARIAL DRUG, MALARIA TREATMENT METHOD, SCREENING METHOD FOR CANDIDATE SUBSTANCE FOR MALARIA TREATMENT, MALARIA SEVERITY MARKER, METHOD FOR TESTING RISK OF SEVERE MALARIA, AND TEST REAGENT

(71) Applicant: OSAKA UNIVERSITY, Suita-shi, Osaka (JP)

(72) Inventors: Hisashi ARASE, Osaka (JP); Fumiji HIRAYASU, Osaka (JP); Kouyuki HIRAYASU, Osaka (JP)

(21) Appl. No.: 16/767,929

(22) PCT Filed: Nov. 28, 2018
(86) PCT No.: PCT/JP2018/043698

§ 371 Date: May 28, 2020

(30) Foreign Application Priority Data
Nov. 28, 2017 (JP) 2017-228226

(51) Int. Cl. A61K 31/7088; G01N 33/15; C07K 14/445; C07K 14/705; A61P 33/06

(52) U.S. Cl. CPC A61K 31/7088; G01N 33/15; A61P 33/06; C07K 14/705; C07K 14/445

(57) ABSTRACT

The present invention provides a new antimalarial drug. The antimalarial drug of the present invention includes: a binding inhibitor that inhibits binding between a RIFIN protein and a leukocyte immunoglobulin-like receptor subfamily B member 1 (LILRB1) protein; an inducer of the binding inhibitor; or an expression inhibitor of RIFIN or LILRB1.

### TECHNICAL FIELD

The present invention relates to an antimalarial drug, a malaria treatment method, a screening method for a candidate substance for malaria treatment, a malaria severity marker, a method for testing a risk of severe malaria, and a test reagent.

### BACKGROUND ART

Vaccines are being developed to prevent malaria infection and to inhibit the symptoms from becoming severe when infected. However, currently, no clinically effective malaria vaccine has been developed.

### SUMMARY OF INVENTION

### Technical Problem

Accordingly, it is an object of the present invention to provide a new antimalarial drug.

### Solution to Problem

In order to achieve the above object, the present invention provides an antimalarial drug including: a binding inhibitor that inhibits binding between a RIFIN protein and a leukocyte immunoglobulin-like receptor subfamily B member 1 (LILRB1) protein; an inducer of the binding inhibitor; or an expression inhibitor of RIFIN or LILRB1.

The present invention also provides a malaria treatment method (hereinafter, also referred to as the “treatment method”), including: administering the antimalarial drug according to the present invention to a patient.

The present invention also provides a method for screening a candidate substance for malaria treatment (hereinafter, also referred to as the “screening method”), including: selecting, as a candidate substance for malaria treatment, a binding inhibitor that inhibits binding between a RIFIN protein and a leukocyte immunoglobulin-like receptor subfamily B member 1 (LILRB1) protein, an inducer of the binding inhibitor; or an expression inhibitor of RIFIN or LILRB1 from a test substance.

The present invention also provides a malaria severity marker (hereinafter, also referred to as the “marker”), wherein the marker is RIFIN.

The present invention also provides a method for testing a risk of severe malaria (hereinafter, also referred to as the “test method”), including: measuring an expression of RIFIN in a biological sample of a subject.

The present invention also provides a test reagent for use in the test method according to the present invention, including: a reagent for measuring an expression of RIFIN.

### Advantageous Effects of Invention

The present invention can provide a new antimalarial drug.

### BRIEF DESCRIPTION OF DRAWINGS

FIG. 1 shows dot plots illustrating the results of flow cytometry in Example 1.

FIG. 2 shows dot plots illustrating the results of flow cytometry in Example 2.

FIG. 3 shows dot plots illustrating the results of flow cytometry in Example 3.

FIG. 4 shows histograms illustrating the results of flow cytometry in Example 4.

FIG. 5 shows histograms illustrating the results of flow cytometry in Example 5.

FIG. 6 shows histograms illustrating the results of flow cytometry in Example 6.

FIG. 7 shows graphs illustrating the results of flow cytometry in Example 7.

FIG. 8 shows graphs illustrating the production amount of IgM in Example 8.

FIG. 9 is a graph illustrating the activity of NK cells in Example 9.

FIG. 10 is a graph illustrating the number of infected erythrocytes binding to LILRB1-Fc in Example 10.

FIG. 11 is a graph illustrating the percentage of Tanzanians with RIFIN protein-binding IgG in Example 11.

FIG. 12 shows histograms illustrating the results of flow cytometry in Example 12.

FIG. 13 is a graph illustrating the results of flow cytometry in Example 12.

### DESCRIPTION OF EMBODIMENTS

In the present invention, the meaning of “treatment” includes avoidance (prevention), inhibition (arrest), or suppression of the progression of symptoms; avoidance (prevention), inhibition (arrest), or suppression from becoming severe; improvement or amelioration of symptoms; or improvement of prognosis. The “treatment” may be any of these meanings.

In the present invention, the meaning of “becoming severe” includes cerebral malaria and severe anemia, and may mean either or both. The cerebral malaria is defined, for example, as follows: Blantyre coma score<3. The severe anaemia is defined, for example, as follows: blood haemoglobin<5 g/dl. The Blantyre coma score is, for example, the sum of the following endpoints (a), (b), and (c). The method for measuring the blood haemoglobin level is, for example, a cyanmethaemoglobin method.

(Endpoints)

### (a) Best Motor Response

Awareness of the site of pain (rubbing the ribs with the finger joints): Score 2
Retract the extremities against the pain stimulus (press the thumbnail bed with the pen): Score 1
No reaction: Score 0

### (b) Verbal Response

Appropriate crying: Score 2
Moan or inadequate crying: Score 1
No reaction: Score 0

### (c) Eye Movement

Move line of sight (follow mother's face): Score 1
Do not move line of sight: Score 0

The present invention will be described below with reference to examples. The present invention, however, is not limited to the following description. In addition, regarding the descriptions of the respective inventions, reference can be made to each other unless otherwise stated.

### <Antimalarial Drug>

As described above, the antimalarial drug of the present invention includes: a binding inhibitor that inhibits binding between a RIFIN protein and a leukocyte immunoglobulin-like receptor subfamily B member 1 (LILRB1) protein; an inducer of the binding inhibitor; or an expression inhibitor of RIFIN or LILRB1. The antimalarial drug of the present invention is characterized in that it includes a binding inhibitor that inhibits binding between a RIFIN protein and a LILRB1 protein, an inducer of the binding inhibitor, or an expression inhibitor of RIFIN or LILRB1, and other configurations and conditions are not particularly limited. According to the antimalarial drug of the present invention, malaria can be treated. As will be described below, the antimalarial drug of the present invention can avoid (prevent), for example, malaria from becoming severe. Thus, the antimalarial drug of the present invention can be referred to as, for example, a drug for avoiding (preventing) malaria from becoming severe.

As a result of intensive studies, the inventors of the present invention have found that RIFIN expressed in infected erythrocytes infected with Plasmodium falciparum binds to LILRB1 expressed in immune system cells such as B cells and NK cells and suppresses the function of the immune system cells. The inventors of the present invention have also found that binding of LILRB1 protein, i.e., expression of LILRB1-binding RIFIN protein is higher in erythrocytes of severe malaria patients than in erythrocytes of mild malaria patients (non-severe malaria patients). Furthermore, the inventors of the present invention have found that, causing a binding inhibitor that inhibits the binding between a RIFIN protein and a LILRB1 protein to coexist, generation of a signal through a LILRB1 protein by a RIFIN protein can be inhibited. From these findings, the inventors of the present invention have found that Plasmodium falciparum evades the immune system through the binding between a RIFIN protein and a LILRB1 protein and causes malaria to become severe, and have established the present invention. According to the antimalarial drug of the present invention, for example, the binding between a RIFIN protein and a LILRB1 protein can be directly or indirectly inhibited, so that the generation of a signal through a LILRB1 protein by a RIFIN protein can be inhibited. Therefore, according to the antimalarial drug of the present invention, for example, the suppression of the function of the immune system cells by the binding between a RIFIN protein and a LILRB1 protein can be prevented or released. Therefore, the antimalarial drug of the present invention can treat malaria, such as by preventing malaria from becoming severe, for example.

In the present invention, RIFIN is derived from Plasmodium falciparum, for example. The RIFIN derived from Plasmodium falciparum can be referred to, for example, from information registered in an existing database. Specifically, examples of the protein of RIFIN of Plasmodium falciparum include the following amino acid sequences. The character strings shown after “Genbank:” in parentheses are the accession numbers in the Genbank of the respective RIFIN proteins (hereinafter, the same applies). Examples of mRNA of RIFIN of Plasmodium falciparum include base sequences registered with the NCBI accession numbers shown in Table 1 below.

TABLE 1

| Gene name | | mRNA |
|---|---|---|
| (Gene ID) | Protein accession Nos. | accession Nos. |
| PF3D7_0100200 | CAB89210.1 (SEQ ID NO: 1) | XM_001350901 |
| PF3D7_0900200 | CAD51688.1 (SEQ ID NO: 2) | XM_001351842 |
| PF3D7_0223100 | CZT98243.1 (SEQ ID NO: 3) | XM_001349697 |
| PF3D7_1254800 | CZT99701.1 (SEQ ID NO: 4) | XM_001350895 |
| PF3D7_0700200 | CZT62652.1 (SEQ ID NO: 5) | XM_002808859 |
| PF3D7_1100400 | CZT98672.1 (SEQ ID NO: 6) | XM_001347658 |
| PF3D7_1480000 | CZU00495.1 (SEQ ID NO: 7) | XM_001348909 |
| PF3D7_0600300 | CAG25174.1 (SEQ ID NO: 8) | XM_960905 |
| PF3D7_0632700 | CAG25139.1 (SEQ ID NO: 9) | XM_961213 |
| PF3D7_0632200 | CAG25134.1 (SEQ ID NO: 10) | XM_961209 |
| PF3D7_0100400 | CAB89212.1 (SEQ ID NO: 11) | XM_001350903 |
| PF3D7_1254400 | CZT99697.1 (SEQ ID NO: 12) | XM_001350891 |
| PF3D7_0732900 | CAD51067.1 (SEQ ID NO: 13) | XM_001349182 |
| PF3D7_1040500 | CZT98660.1 (SEQ ID NO: 14) | XM_001347646 |
| PF3D7_0600500 | CAG25176.1 (SEQ ID NO: 15) | XM_960907 |
| PF3D7_1254200 | CZT99695.1 (SEQ ID NO: 16) | XM_001350889 |
| PF3D7_0400700 | CAD49098.1 (SEQ ID NO: 17) | XM_001351287 |
| PF3D7_1040700 | CZT98662.1 (SEQ ID NO: 18) | XM_001347648 |
| PF3D7_0500400 | CAD51370.1 (SEQ ID NO: 19) | XM_001351529 |
| PF3D7_1255100 | CZT99704.1 (SEQ ID NO: 20) | XM_001350898 |
| PF3D7_1300400 | CAD52146.1 (SEQ ID NO: 21) | XM_001349705 |
| PF3D7_0732200 | CAD51061.1 (SEQ ID NO: 22) | XM_001349176 |
| PF3D7_0115600 | CAD48968.1 (SEQ ID NO: 23) | XM_001351042 |
| PF3D7_1101100 | CZT98679.1 (SEQ ID NO: 24) | XM_001347665 |
| PF3D7_0401400 | CAD49104.1 (SEQ ID NO: 25) | XM_001351293 |
| PF3D7_1000200 | CZT98249.1 (SEQ ID NO: 26) | XM_001347253 |
| PF3D7_0937500 | CAD52049.1 (SEQ ID NO: 27) | XM_001352203 |
| PF3D7_1000500 | CZT98252.1 (SEQ ID NO: 28) | XM_001347256 |
| PF3D7_1479700 | CZU00492.1 (SEQ ID NO: 29) | XM_001348906 |
| PF3D7_0632400 | CAG25136.1 (SEQ ID NO: 30) | XM_961211 |
| PF3D7_0101000 | CAX51180.1 (SEQ ID NO: 31) | XM_002808552 |
| PF3D7_1400600 | CZT99711.1 (SEQ ID NO: 32) | XM_001348145 |
| PF3D7_1040300 | CZT98658.1 (SEQ ID NO: 33) | XM_001347644 |

Plasmodium falciparum RIFIN protein 1
(SEQ ID NO: 1, PF3D7_0100200, GenBank: CAB89210.1)
MKIHYINILLFELPLNILIYNQRNHKSTTPHTPNHTQTTRLLCECELYSPANNDNDAEMKR
VMQQFEDRTTQRFHEYDERMKTTRQKCKEQCDKEIQKIILKDKLEKELMDKFATLQTDI
QSDSIPTCICEKSLEDKVEKGCLRCAGVLGGGIAPGWSLVSGLGYAVWTNYVTQTALQK
GIEAGVKAGIEGLRDFSGLGKLIPISVIQNLINHTNYDIAKTYITFVKSVNSTKCAVKEHSF
CFSTYISNENALSKRAAGIAEYAADMAKITERGVLDAATPGLTTYSNAITASVVAIVVIVL
VMIIIYLILRYRRKKKMKKKLQYIKLLEE
Plasmodium falciparum RIFIN protein 2
(SEQ ID NO: 2, PF3D7_0900200, GenBank: CAD51688.1)
MKVHYINILLFALPLNILIYNQRNHNSTTHHTLKIPITRLLCECELYELANYDNDPEMKEV
MQQFEDRTTQRFHEYDERMKTTRQKCKDKCDKEIQKIILKDKLEKELMDKFATLHTDIQ
SDAIPTCICEKSLEDKMEKECLKCAQNLGGIVAPSTGVLGEIAALAVNAWKTEAIAAATK
AAIAKGTAKGLAAGAAKGVAEVIAQVESQFRLSTIGVKELGSIFNASNYTNETFISGYIYA
QYQGSQCGSLSMLLGKSKPFCTFVEGRIFATSVRVGRSFSPEDFIKTTVQTIVKNAKTTAE
ATKAQVASAEKAAVLETSKKAIEATTTPYYTPIIVSIVAIVVIILIMVIIYKILRYRRKKKMK
KKLQYIKLLKE
Plasmodium falciparum RIFIN protein 3
(SEQ ID NO: 3, PF3D7_0223100, GenBank: CZT98243.1)
MKDHYINILLFALPLNILVYNQRNYYITRTPKATTRTLCECELYAPATYDDDPQMKEVMD
NFNRQTQQRFHEYDERMKTTRQKCKDQFDKEIQKIILKDKLEKELMDKFATLQTDIQND
AIPTCICEKSLADKVEKTCLRCGSVFGGGITPGWGLISGLGYVGWTNYITEIAIQKGIEAG
VKAGIQELKGFAGLSRLINFSEIKNLINHTNYFKEMTYVSFLQDANKTHCSARPTSKEIFC
NFVSHNGESALSKRAAGIADYAADMAKITEEGVLEEGASATSSLTTAIIASIIAIVVIILIMII
IYLVLRYLRKKKMKKKLEYIKLLKE
Plasmodium falciparum RIFIN protein 4
(SEQ ID NO: 4, PF3D7_1254800, GenBank: CZT99701.1)
MKIHYTNILLFPLKLNILVNTHKKPSITPRHIQTTRLLCECELYMSNYDNDPEMKRVMQQ
FHDRTTQRFHEYDDRMIEKRQKCKDRCNKEIEKIILKDKIEKELTETFATLNTNITNEDIPT
CICKKSVADKIEKTCLKYGGALGGGVMPGLGLIGGNSVYILANYETINAFIAKTIEELEGI
PGITKLFGAKISQFVTPAVFRKPMSLVETILSEKKKLCLCAANKNELLCRGMNPNVPETLP
KKIEVAVNEVLSSVNDTWATATTPTTFFTNPIILSAIAILVIVIIMVIIYLILRYRRKQKIKKKL
QYIKLLKE
Plasmodium falciparum RIFIN protein 5
(SEQ ID NO: 5, PF3D7_0700200, GenBank: CZT62652.1)
MKIHYINILLFALPLNILVHNQRNHKKTILHTPKTKSTRTHRSLCECELYAPVNYYSDPQM
KEVMDNFNKQTQQRFHEYDERMKTTRQKCKDRCDKDIQKIILKDKIEKELAETFSSLHT
DIQSDAIPTCICEKSLADKVEKGCLRCAQNLGGLVPGMGLIGGTAVYAAAVKAATKAGM
KEALEGLKSIGGLKLLLQDKFTELVTTRNFQCPNALVGAVQNVINTQCVGPAAKNQLLC
NGYEAQNDSRIIQKAVDAGRDGADVYIRTFSDSTTITTFLTDPIVISAIVVISIVVILLIIYLIL
RYRRKIKMNKKLQYIKLLKE
Plasmodium falciparum RIFIN protein 6
(SEQ ID NO: 6, PF3D7_1100400, GenBank: CZT98672.1)
MKIHYINILLFALPLNILVNNQRNHNNSTYHTSNTKTIKSHRSLCECELYAQSNYENDQE
MKDVIKEFNDRTAQRFEEYNERMQVKKDQCKEQCDKEIQQIILKDKIEKELTERFSALET
KIDTNDILTCICEKSVTDKFEKTCLKCSGIFATAVPELGLIGGTVVYAAAVKAATKAGMEA
ALVGLESVNGLRGLLGEKIKDLVTTTNFQCPNALMGLVQNVKDTQCVGAAAQSQVFCK
GLLPESTSRIIQKAAAAGREGAEAYNTTFSDSTTITAFLTDPIVISAIVVISIVVILLIIYLILRY
RRKIKMNKKLQYIKLLKE
Plasmodium falciparum RIFIN protein 7
(SEQ ID NO: 7, PF3D7_1480000, GenBank: CZU00495.1)
MKVHYINILLFSLPLNILEHNPWNHYMKPHTYTNRSLCECELYELANYDNDPQMKEVM
ENFIKQTQQRFHEYDERLQSKRKQYKDKCDKEIQKIILKDKLEKQMAQQLTTLDPNITTE
DIPTCVCEKSLADKTEKFCLNCGKTMGGVAPGWGLVSGLGYAGWSHYAATTLVKIATD
AGIAEGLKVGLTKVTEIVTQLSSSTEVAIPTIDVLTNLTTGISADNVTLLGIFKTINTGMKGE
FDTDTYALFSTWVQNIATTPKSYMGRYLTEAEEVTKAFADAQTRVLTQAGNVTSNLTTGI
TVSIIAIVVIVLVMLITYLILRYRRKKKMKKKLQYIKLLKE
Plasmodium falciparum RIFIN protein 8
(SEQ ID NO: 8, PF3D7_0600300, GenBank: CAG25174.1)
MKIHYINILLFELPLNILIYNQRNHNSTTPHHPPNTRLLCECELYAPATYDDDPQMKEVMQ
QFEDRTSQRFHEYDERMKTTRQKCKDKCDKEIQKIILKDKLEKELMDKFATLQTDIQND
AIPTCVCEKSLEDKMEKGCLRCGGVLGGGIAPTFGLIGSVAINMWKTTEIAAATKAAIAA
GKAAGKIAGEAAGKKAVIEALKYFGVDDFFPEIFKSILKMSRYTDVTKFGAAIAEKHVL
NCAMSARGGSVNDSTCNAFEIKLGLFEAETGKPNGPPAYQAIPQKINELAEEATQAAAEA
AKKASESATAAFETAEKEAIEAASMQLYTTIAYSILAILIIVLIMVITYLILRYRRKKKMKKK
LQYIKLLEE
Plasmodium falciparum RIFIN protein 9
(SEQ ID NO: 9, PF3D7_0632700, GenBank: CAG25139.1)
MKIHYINILLFELPLNILIYNQRNHYITRTPKATTRSLSECELYAPSNYDNDPQMKEVMDN
FNRQTQQRFHEYDHRMKTTRQKCKEQCDKEIQKIILKDKLEKELMDKFATLHTDIQNDA
IPTCVCEKSVADKVEKNCMKCTQNLGGIVAPSSGVLAGIAEGALYVWRDAEIVAAIAAA
KEAGAAKGAAVGIKEGIKVLLNRLNTDFGLSPVRIKELESVINGTNYTDVTFIYEAIYTTY
KRSCVPVDVSVRFTVADTDLTFCESVWNQTLAVSQRNMGTSPLPIIQKTAQKIVSDANFT
AAATAETATEEATTTLTAKNTGEVNATYMGYQTPIIASIVAILVIVLVMIIIYLILRYRRKKK
MKKKLQYIKLLEE
Plasmodium falciparum RIFIN protein 10
(SEQ ID NO: 10, PF3D7_0632200, GenBank: CAG25134.1)
MKIHYTNILLFPLKLNILVNTHQKPHTTARHTQKIPTTRSLSECELYAPVNYYSDPQMKEV
MDNFNKQTQQRFHEYDERVQNTRQKCKEQCDKEIQKIILKDKIEKELNEKFSALHTDIQS
DDIPTCICEMSMADKVEKGCLRCVGVFGGGIAPSVGLLGGLGIYVWKPGALKVAITAAL
NANSVKIAAAANAAGEAMGVKTVIEGLKALNVHGLCPDLFESIGTKIHYTNAEEIAKIIV
AKYRATCNLSTGTSSTQAMCKQFDYTFGMRIRLGSPVEYGPPPASAIPDTVKKVVAGAE
QAAEAKAANVRTTISSKIITEETDVINTIYMSNQTAIIASIIAIVIIVLIMVIIYLILRYRRKKK
MKKKLQYIKLLEE
Plasmodium falciparum RIFIN protein 11
(SEQ ID NO: 11, PF3D7_0100400, GenBank: CAB89212.1)
MKIHYTNILLFPLKLNILVNTHKKPSITSRHIQTTRLLCECELYTPNYDNDPEMKSVMQQF
HDRTTQRFHEYDENLKEKRQKCKDKCDKEIQKIILKDKIEKELTEKFSSLQTDITISDAIPT
CICEKSLADKVEKNCLKCTQNLGKIVAPSSGVLAGISEAALSVWKTTEIAAAMELAKQA
GAAAGLKAGHLAGTNAVIEQLRTLGIYFVGDKLLETIIDVTNYMNVSFIYDKVYSHYTTS
CTPSLVNDQLVGTFNTSDPFCNLVHSNLQGSFYRSSAQTIIYEKVEEAVAGAEQAATTKTA
VMTPIYTTEFTAKNIAEVEAATTSYYTPIIASIVAIVIIVLIMVIIYLILRYRRKMKLKKKLQ
YIKLLEE
Plasmodium falciparum RIFIN protein 12
(SEQ ID NO: 12, PF3D7_1254400, GenBank: CZT99697.1)
MMLNYTNILLFYLSLNILSSSSEVYNQRNHFITYTPKRSTRLLCECELYTSIFDNDPEMKS
LIEHFNKQTQQRFHEYDERMKTTRQKCREQCDKEIQKIILKDKLEKELAEKFVTLQTDIQ
SDAIPTCICEKSLADKVEKTCLKCGGVLGGGVTPAWGFLSGIVYTGWKAAALAAATKEA
IAEGAAKGAAAGTKAGIKAVMDVLYSDFGLSIEGVQKMGLVLSATNYKDVPMITKALYS
KFQVSSCLRGGPVPGVPPVRPTDGTFCSAMLEKILAQENVVKQNSLEGSIKSVVNQIVTE
AKSAAVSETAKVTASETETLKATNIAAVNATYASSQTAIIASIIAIVVIILIMVIIYLILRYRRK
KKMKKKLQYIKLLEE
Plasmodium falciparum RIFIN protein 13
(SEQ ID NO: 13, PF3D7_0732900, GenBank: CAD51067.1)
MKIHYINILLFELPLNILIYNQRNHNSTTHHTLKIPITRLLCECELYAPSNYDNDPEMKEVM
EIFDRQTSERFHEYDERMKTTRQKCREQCDKEIEKIILKDKLEKELMDKFATLHTDMQSD
AIPTCVCEKSVADKTEKVCLNCGKTMGAVAPAWGLISGLWYATWSQYVSAKILEVGISE
GIKEGLTQIMKFTISLYPKANLPNITVTQMLSSGKFTNNVTLFDMVQHINNTMYTTLEAE
EYSKFCGVVSSMAKYKNITFNRTYGKYSTAVTEAVTQGKTNAINTLTPATNTLTTAIIASM
VAIVVIVLVMIIIYLILRYRRKKKMKKKLQYIKLLEE
Plasmodium falciparum RIFIN protein 14
(SEQ ID NO: 14, PF3D7_1040500, GenBank: CZT98660.1)
MKVHYINILLFVIPLNILINDQRNHKSTTHHTLKIPITRLLCECELYTPANYDNDPQMKEV
MDNFNRQTQQRFHEYDERMVEKRMQCKDKCDKEIQKIILKDKLEKELMDKFATLHTDI
QSDAIPTCVCEKSVADKMEKGCLRCGSILGAAMPEMGSIGGSLLSALSAWKPVAIEAAE
KAAIAKATDLATQAGMREVVLKIEQFLKNFTEKEGLVNFTSVVNKSNFKCPTALFQNAN
ELLSDSCIPDEVTNRTSTFCSTIAYGEKTTFEPFAQAGATTFQETLTAKTPVLQARYTAAVK
TAYGGYQTAIIASIVAIVVIVLIMVIIYKILRYRRKKKMKKKLQYIKLLEE
Plasmodium falciparum RIFIN protein 15
(SEQ ID NO: 15, PF3D7_0600500, GenBank: CAG25176.1)
MKIHYTNILLFALPLNILVNTHKKPHTTARHTQKIPTTRSLSECELYAPVNYYSDPQMKEV
MDNFNKQTQQRFHEYDERMKTTRQKCKDKCDKEIQKIILKDKLEKELMDKFATLDTDI
QSDAIPSCVCEKSIAEKAEKGCLRCGYGLGSVAPMIGLTGSVAVNVWKTAELAAAMELA
KQAGAAAGIKAGHLAGTKVVIDQLHTLGIYFVGGKPLESIIHVTNYMNVSVIYDKVYSH
YTTLCTPRFVIDRPVGDFIFSGPVCNLVQPNHQGIWVKSSAQAIIKKKVEEAVAEGTQAA
DVVAKNTADEVTKAAIKTSTEAIDAATTTYYTPIIASIVAIVLIVLIMVITYKILRYRRKRKM
KKKLQYIKLLEE
Plasmodium falciparum RIFIN protein 16
(SEQ ID NO: 16, PF3D7_1254200, GenBank: CZT99695.1)
MKVHYINILLFALPLNILIYNQRNHKSTTHHTLKIPITRLLCECELYTPANYDNDPQMKEV
MDNFNRQTQQRFHEYDERMVEKRMQCKDKCDKEIQKIILKDKLEKQMEQQLTTLETKI
DTNDIPTCVCEKSMTDKVEKGCLRCGRNLGVAVPGLGVLGAYGAHSIVKVAMATAEKV
GIQLGIDAGNAAGIKAVIEALNSSLNIDNLGGITLDTVLKGNNFKNIDFLVYILTDKYNTT
CTVSNTEVETLLCYIGKEKPTLPYTLIQSNVRKAVAEATEVATSTTEEMTTIYTTQELSKV
TSTGAILSNPIIISFIVIVIVVIIFLITYLILRYRRKKKTKKKLQYIKLLKE
Plasmodium falciparum RIFIN protein 17
(SEQ ID NO: 17, PF3D7_0400700, GenBank: CAD49098.1)
MKIHYINILLFELPLNILIYNQRNHKSTTHHTLKIPTTRLLCECELYSPANYDNDPEMKEV
MEIFDRQTSERFHEYDERMVEKRMQCKDKCDKEIQKIILKDKLEKELAEKFVTLQTDIQ
NDAIPTCVCEKSIADKVEKGCLRCVGVFGGGVMPGFGTIGGTALYALNQLKPAVFKAAI
KAALEEGAAEILAAGIEAGDAAGMNVVRYGLRYLHVHELFPVIFDSFVKTRPYNEITSIA
NSILLKYGPTCTGLDNNSPPAACTKFQLNLGIHKKIGAMIDTHGTPASTAIRQGLEGILEE
ATQTAEAAAKIAEKGVAAEITARETALIEAGFNSSITSINASIFAIVVIVLIMVIIYLILRYRR
KKKMKKKLQYIKLLEE
Plasmodium falciparum RIFIN protein 18
(SEQ ID NO: 18, PF3D7_1040700, GenBank: CZT98662.1)
MKFSYFNILLFSIPLNILINDQRNHKSTTHHTLKIPITRLLCECELYSPDNYDNDAEMKRV
MQQFEDRTSQRFHEYDERMQSKRMQCKDRCDKEIQKIILKDKIEKELSQHLSTLETNIDT
NDIPTCVCEKSLADKVEKGCLRCGYGLGTVAPTVGLIGAVAVNELKKAAMAIAIKDAIAE
GLVAGETARIQASIKAVILGIKSKFRIDTLGGEVLESIITAQKYDDVSLISESIYMQYQSTCL
PQYVGHGADLSKPICHTVYTLDFVQGKVHVPGSLQGSIKKALEKIVAEAKSNAVSETAN
VTTRQTAVFESRNIAAVDATYASYQTAIVASVVAILVIVLVMLIIYLILRYRRKKKMKKKLQ
YIKLLKE
Plasmodium falciparum RIFIN protein 19
(SEQ ID NO: 19, PF3D7_0500400, GenBank: CAD51370.1)
MKFSYFNILLFSIPLNILINDHSKYSSCKHTSNSKTTKPHRSLYECGLYSPANNDNDPEMK
RVMQQFEDRTSQRFHEYDERMQSKRMQCKEQCDKEIQKIILKDKLEKQMEQQLNTLET
KIDTDDIPTCVCEKSLADKVEKGCLRCGYGLGTVAPTVGLIGAVAVHVWKPMALEAAIE
AAIAKSAAEISAAANAAGIQAGKIAVIESLKKLYVDYFWPEMSNYILNMSHYNGVANLTA
FIHEPKFNVCKDAGEVILDKCNAFDMGFGILKKDGVTNGLLPKDAVPRVLKGIVGQAEG
PAKVAADAARQTVTAEITEKETAAINTIFMSKQTAIIASVVAIVVIVLIMIIIYLILRYRRKK
KMKKKLQYIKLLKE
Plasmodium falciparum RIFIN protein 20
(SEQ ID NO: 20, PF3D7_1255100, GenBank: CZT99704.1)
MKIHYINILLFPLKLNILIYNQRNHKSTTHHTLKIPITRLLCECELYTPANYDNDPQMKEV
MDNFNRQTQQRFHEYDERMVEKRMQCKDKCDKEIQKIILKDKLEKQMEQQLTTLETKI
TTDDIPTCLCEKSVADKMEKTCLRCAGVLGGGVMPGMGLIDGSLLGAISVLKPAAIIAAK
DAALAEATALATQAGMREVVLKIEQFLKLFSEKEKIFDLKLIVNKSNFSCGSSLFQNAKE
LANKSCVAKPNGSYTSFCNSITYSRVEPFNGYAQAGITKYNETLPLQKALLEKAKVDAVN
TTYAAYHTSIIASIVAVVVIVLIMVIIYLILRYRRKKKMKKKLQYIKLLEE
Plasmodium falciparum RIFIN protein 21
(SEQ ID NO: 21, PF3D7_1300400, GenBank: CAD52146.1)
MKIHYTNILLFPLKLNILVNTHKKPHTTARHTQKIPTTRSLSECELYAPVNYYSDPQMKEV
MDNFNKQTQQRFHEYDERMKTTRQKCKDKCDKEIQKIILKDKLEKQMAQQFSTLHTDI
QSDDIPTCICEKSLADKVEKGCLRCAQNLGGVAPGWGLLSGFGYVTWSQYISGIAAKAA
ADAGLKAGVKVGLVNAVKIVTKTLDGFGEVPTMDWAKLIAFGDFSDGVTLHAIFKNLN
NMMNCYLDSGKYSQFSTVVQKFAENPRSYATPYSTEVTEVTKAVADAKTGVLTKAGNA
TSSLSTGITASIIAIVVIVLIMVIIYLVLRYRRKKKMKKKLQYIKLLEE
Plasmodium falciparum RIFIN protein 22
(SEQ ID NO: 22, PF3D7_0732200, GenBank: CAD51061.1)
MKDHYINILLFALPLNILVYNQRNYYITPRHTETNRSLCECELYSPTNYDSDPEMKRVMQ
QFVDRTTQRFHEYDERMKTTRQKCKDKCHKEIEKIILKDKMEKQMAQQLTTLETKIGTD
DIPTCVCEKSMADKMEKDCLRCTYGLGTLAPTVGLIGSVAVGAWKPTALKAAIVAAQK
AGDAAGVAAGEAAGKKAVILALQHFKLDNLFPEIYNAIVKIRHYADVKNFSVAIVEEHSL
KCQSLDLKVTTNPTCETFEFNIGMRIPDSSFVEPVDQVVPEVLDSLVGNIKEVAEAKAAE
VAAAKTAEFKIANVGAVESTYGSCQTAIIASIVAIVVIVLIMVIIYLILRYRRKKKMKKKLQ
YIKLLKE
Plasmodium falciparum RIFIN protein 23
(SEQ ID NO: 23, PF3D7_0115600, GenBank: CAD48968. 1)
MKVHYINILLFALPLNILIYNQRNHKSTTHHTLKIPITRLLCECDIYTSIYDNDPQMKEVM
DNFNRQTQQRFHEYDERMQGKRQKCKDKCDKEIQKIILKDKLEKELMDKFATLHTDMQ
SDSIPTCVCEKSVADKVEKNYMKCTQNLGGIVAPSSGVLAGIAELGLSAWKTTALKTAIA
AAEQAGAAKGLAAGAAKGATRLIELIQSTFKIQNIAGKSLGTFIDATNYNNGPFIYQAIYT
KFEMSLCLPVFPGVDPVPGAVRDPTFCNLFEKFVPTNGSSNRDSIINAIETYVQPFVSDAK
FTAAATAETATEEATAVLITKKTGEVTTTYASYQTAIIASIVAILVIVLVMIIIYLILRYRRKK
KMKKKLQYIKLLEE
Plasmodium falciparum RIFIN protein 24
(SEQ ID NO: 24, PF3D7_1101100, GenBank: CZT98679.1)
MKIHYINILLFELPLNILIYNQRNHKSTTHHTLKIPITRLLCECELYAPSNYDNDPEMKEVM
EIFDRQTSERFHEYDERMKTTRQKCKDKCDKEIQKIILKDKLEKELNEKFLTLQTDIQND
AIPTCVCEKSLADKVEKGCLRCGSILGAAMPEVGSIGGGLLYALNAWKPKALEAAIAAA
KELAITEATNAGVKTVVSEINKLLAKFKQHEILFELKPIVNKSNFSCGSSLFQRAEELASK
SCVAQPNGSYTSFCNTILNGEKTTFKPFAQAGANTYEKTLTTETPVLQARYTAAVKTAYG
GYQTAIIASIVAIVVIVLIMVIIYLILRYRRKKKMKKKLQYIKLLEE
Plasmodium falciparum RIFIN protein 25
(SEQ ID NO: 25, PF3D7_0401400, GenBank: CAD49104.1)
MKVHYINILLFALPLNILVINQRNHNNSTYHTSNTKLTKTHRTLCECELYAPSNYENDPEM
KELMENFNHQSSERFREYDERIQDKRKQFKEQCEKDIQKIILKDKIEKELTEKLSTLQTDI
STNDIPTCVCEKSLADKMEKTCLKCGGVLGTAVPELGLIGGSVIYSAAQAAAAKLGVAK
AIELMKKIYNLGNVSFIDWTNLINVGNYSHRMSLVGIVNKVNNMCQIKDPEGNVVFCFA
KQNMRGGAGKFAQTISEQAGNAAIKAGETANVKFAEMTSVGTIFSDPIVISATVVVTIAVI
LIIIYLILRYRRKKKMKKKLQYIKLLEE
Plasmodium falciparum RIFIN protein 26
(SEQ ID NO: 26, PF3D7_1000200, GenBank: CZT98249.1)
MKIHYINILLFELPLNILIYNQRNYYITPRHTETNRSLCECELYSPTNYDNDPEMKRVMQQ
FVDRTTQRFHEYDERMKTTRQKCKERCDKEIQKIILKHKLEKELMDKFATLHTDIQSDAI
PTCVCEKSLADKTEKFCHNCGYGLGSVAPNIGLLGGPGIYVWKIAALAAAKEFAEKAGA
AMGKAAGDAAGAAELIRGLKALNIDKLFNESLGLVFDGTNYNNTEYIFKAIFSKFNESC
MPRPPGSVPGPVIDRAFCDTVDTLVLPSGTGSQTSASTNAVIKEYVKPIVSNAKFTAEATA
QTAAEEATNLALKTNTNAVNATYASSQTAIIVSIAAIVVIVLVMIIIYLILRYRRKKKMKKK
LQYIKLLEE
Plasmodium falciparum RIFIN protein 27
(SEQ ID NO: 27, PF3D7_0937500, GenBank: CAD52049.1)
MKIHYTNILLFPLKLNILVNTHKKPSITARHIQTTRLLCECELFSPQNYDNDPEMKRVMQQ
FHDRTTQRFHEYDERMKTTRQECKEQCDKEIQKIILKDKMEKQMAEKLSTLETKINTDD
IPTCVCEKSMADKTEKFCLNCGKNMAAIAPWWGLVCGSGYAGWLHSAMAAAIDKAIA
EGAAAGIKAGHLAGTNAVIEQLRTLGIYFVGNKQLETIIDVTNYMNVSFIYDKVYSHYIT
LCTPRPVNGHLVSNFNFSDRFCKLFHQKDLVSLDIKSVKAIIKKNVEEAVAGAEQAAKAE
VSNVTATKTTEFTTKNIAEVEAATTSYYTPIIASIVAIVIIVLIMVIIYKILRYRRKKKMKKK
LQYIKLLEE
Plasmodium falciparum RIFIN protein 28
(SEQ ID NO: 28, PF3D7_1000500, GenBank: CZT98252.1)
MKVHYFNILLFALPLNILVSSPKKNPSITQKRPTRRLLCECELYAPANYDSVPQMKEVMD
NFNRQTQQRFHEYDERMVEKRMQCKDKCDKEIQKIILKDKLEKQMKQELTTLETKITTD
DIPTCICEKSLADKVEKGCLRCGGVFGGGVAPGVGLLGGIGQLGLDVWKAAAIKAATEY
ALTEGAAKGLAAGNAHGMNIVIYHLKELLIDKLVPNICKTVSSTGDYTRVINFSKLIIQKR
GAMCGADGGTLSKDMCTQININLGTVLRNGKANLPDKEAVPKVLNRLVSQADKAANE
VAKDTSQSVAVKITEQQTAAINATYTSWQIAITASVIAIVVIVLIMVIIYLILRYRRKKKMK
KKLQYIKLLEE
Plasmodium falciparum RIFIN protein 29
(SEQ ID NO: 29, PF3D7_1479700, GenBank: CZU00492.1)
MKVHYINILLFALPLDILEHNKNEPHTTPNHTQTTRSLCECELYSPANNDNDPEMKRVMQ
QFEDRTSQRFHEYDERMVEKRMQCKDKCDKEIQKIILKDKLEKQMVEQFSTLQTDIQSD
AIPTCVCEKSIEDKVEKGCLRCGSILGAAMPELGSVGGSLLYALNTWKPAAIIAAKEAAL
AEATDLATQAGIDTVVAQLKIEGLLASFTVKQRLVDLSSIVTSSTYNNGAILHKSAMELAS
SYCHFEGTQSTPPFCSTIKYGQTTNFVRYAKAGSAAFKTEFASKSATLTKAKVGAVEATY
GGYHISIISSIVAIVVIVLIMVIIYLILRYRRKKKMKKKLQYIKLLEE
Plasmodium falciparum RIFIN protein 30
(SEQ ID NO: 30, PF3D7_0632400, GenBank: CAG25136.1)
MKVHYINILLFALPLNILIYNQRNHKSTTHHTLKIPTTRLLCECDLYIPNYDNDPQMKKIM
ENFDRQTSQRFHEYDERMKTTRQKCKDKCDKEIQKIILKDKIDKELTEKFATLQTDIQND
AIPTCVCEKSLADKVEKTCLKCGGVLGGGVTPAWGLISGIVYTGWKAAALAAAKKLAA
EAGAAEGASQGAAAGATRLIELIQSTFQVQNIAGQSLESIFTAQTYTDVSNITKALFNEYA
EICLPIFTDSVPVRGVRYNISSPICTFVEEGILATSRDKGGSPITFIEKKVETMVSKAEGVAT
ARAADVAAAKTAEFEATKVGAVEATYAGYHTTIIASIIAILIIVLIMVITYLILRYRRKKKMK
KKLQYIKLLEE
Plasmodium falciparum RIFIN protein 31
(SEQ ID NO: 31, PF3D7_0101000, GenBank: CAX51180.1)
MKVHCYNILLFSFTLIILLLSSSQVNNQMNHYNTAHMKNTEPIKSYRSLCECELYTSMYD
DDPEMKEILHDFDRQTSQRFEEYNERLLENKQKCKEQCEKDIQKIILKDKLEKELMDKF
ATLHTDIQSDAIPTCVCEKSIADKMEKECLRCAQNLGGIVAPSSGVLAGIAEGALIVWKP
AAIKAAKAAAAKAASDAATQAGMNAVRLEIKKLLEMFTGKPGYVDLLPIVKESTYKNG
SALVDSAKKLFVESGKLEGLDRMPVFYNTVIDYPGPSNIKGFGKIGSDAYEAAFTSQKGT
LEATKVGEVNTTYGGCQTAITASVIAIVVIILIMVITYLILRYRRKKKMKKKLQYIKLLEE
Plasmodium falciparum RIFIN protein 32
(SEQ ID NO: 32, PF3D7_1400600, GenBank: CZT99711.1)
MKDHYINILLFALPLNILVYNQRNYYITPRHTETNRSLCECELYSPTNYDSDPEMKRVMQ
QFVDRTTQRFHEYDESLQSKRKQCKDQCDKEIQKIILKDKIEKEFTEKLSTLQTDITTKDI
PTCVCEKSLADKMEKVCLKCAQNLGGIVAPSTGVLGEIAALAVNAWKTTALKNAIAAA
QKAGDAAGKIAGESKGVETIIGILEQYYSIYELKGTPLKSFFATTHYTDISNIATVIDTELN
TSCGLNSLANQAICGLRTKLGLVAKPGQVMVTQKEAITKMITNVVHKSEITAEAAKTEV
AATKTAAAIKMNTEMEAATTPYYTPIIASIVAIVVIVLIMVITYLILRYRRKKKMKKKLQYI
KLLN
Plasmodium falciparum RIFIN protein 33
(SEQ ID NO: 33, PF3D7_1040300, GenBank: CZT98658.1)
MKFNYTNIILFSLSLNILLLSSRVYNKRNHKSIILHTSNENPIKTHRSLCECELYSPTNYDSD
PEMKRVMQQFHDRTTQRFHEYDERMKTTRQECKEQCDKEIQKIILKDRLEKELMDKFAT
LHTDIQSDAIPTCVCEKSLADKTEKFCLNCGVQLGGGVLQASGLLGGIGQLGLDAWKAA
ALVTAKELAEKAGAAAGLKAGDIHGMKIVIEGLKALKVDTLKSGIFNSFVNNSHYTEVT
GLAIAIDTEMNEVCSATYIGIHPICVVREKLGVIPKAGGTMVKQKDAITNVLKQALEKAT
QSAEALSETTAEDVAAKLTAQKTGAINTIFMSNQTAIIASIVAIVVIVLIMVITYLILRYRRK
KKMKKKLQYIKLLEE

The RIFIN preferably include, for example, a LILRB1-binding RIFIN. Examples of the LILRB1-binding RIFIN include the RIFINs described below. Preferable examples of the RIFIN include PF3D7_1254800 (SEQ ID NO: 4), PF3D7_0223100 (SEQ ID NO: 3),

PF3D7_1100400 (SEQ ID NO: 6), PF3D7_0632700 (SEQ ID NO: 9), PF3D7_0700200 (SEQ ID NO: 5), and PF3D7_0100200 (SEQ ID NO: 1) because of their stronger binding to LILRB1. The character strings shown after “Genbank:” in parentheses are the accession numbers in the Genbank of the respective RIFIN proteins.
PF3D7_0100200 (SEQ ID NO: 1, GenBank: CAB89210.1)
PF3D7_0900200 (SEQ ID NO: 2, GenBank: CAD51688.1)
PF3D7_0223100 (SEQ ID NO: 3, GenBank: CZT98243.1)
PF3D7_1254800 (SEQ ID NO: 4, GenBank: CZT99701.1)
PF3D7_0700200 (SEQ ID NO: 5, GenBank: CZT62652.1)
PF3D7_1100400 (SEQ ID NO: 6, GenBank: CZT98672.1)
PF3D7_1480000 (SEQ ID NO: 7, GenBank: CZU00495.1)
PF3D7_0600300 (SEQ ID NO: 8, GenBank: CAG25174.1)
PF3D7_0632700 (SEQ ID NO: 9, GenBank: CAG25139.1)
PF3D7_0632200 (SEQ ID NO: 10, GenBank: CAG25134.1)
PF3D7_0100400 (SEQ ID NO: 11, GenBank: CAB89212.1)
PF3D7_1254400 (SEQ ID NO: 12, GenBank: CZT99697.1)

In the present invention, the origin of leukocyte immunoglobulin-like receptor subfamily B member 1 (LILRB1) is, for example, human. The human-derived LILRB1 can be referred to, for example, from information registered in an existing database. Specific examples of the human-derived LILRB1 include the following base sequence (SEQ ID NO: 34) registered with NCBI accession NO. NM_001081637.2 as an mRNA and the following amino acid sequence (SEQ ID NO: 35) registered with NCBI accession NO. NP_001075106.2 as a protein. It is to be noted that, in the base sequence of LILRB1 mRNA and the amino acid sequence of LILRB1 protein described below, the underlined region is a region corresponding to the extracellular region of LILRB1 used for the preparation of LILRB1-Fc in Examples to be described below.

Human LILRBI mRNA
(SEQ ID NO: 34)
CTCAGCCTGGGCGGCACAGCCAGATGCGAGATGCGTCTCTGCTGATCTGAG
TCTGCCTGCAGCATGGACCTGGGTCTTCCCTGAAGCATCTCCAGGGCTGGA
GGGACGACTGCCATGCACCGAGGGCTCATCCATCCACAGAGCAGGGCAGTG
GGAGGAGACGCCATGACCCCCATCCTCACGGTCCTGATCTGTCTCGGGCTG
AGTCTGGGCCCCCGGACCCACGTGCAGGCAGGGCACCTCCCCAAGCCCACC
CTCTGGGCTGAACCAGGCTCTGTGATCACCCAGGGGAGTCCTGTGACCCTC
AGGTGTCAGGGGGGCCAGGAGACCCAGGAGTACCGTCTATATAGAGAAAAG
AAAACAGCACCCTGGATTACACGGATCCCACAGGAGCTTGTGAAGAAGGGC
CAGTTCCCCATCCCATCCATCACCTGGGAACACACAGGGCGGTATCGCTGT
TACTATGGTAGCGACACTGCAGGCCGCTCAGAGAGCAGTGACCCCCTGGAG
CTGGTGGTGACAGGAGCCTACATCAAACCCACCCTCTCAGCCCAGCCCAGC
CCCGTGGTGAACTCAGGAGGGAATGTAACCCTCCAGTGTGACTCACAGGTG
GCATTTGATGGCTTCATTCTGTGTAAGGAAGGAGAAGATGAACACCCACAA
TGCCTGAACTCCCAGCCCCATGCCCGTGGGTCGTCCCGCGCCATCTTCTCC
GTGGGCCCCGTGAGCCCGAGTCGCAGGTGGTGGTACAGGTGCTATGCTTAT
GACTCGAACTCTCCCTATGAGTGGTCTCTACCCAGTGATCTCCTGGAGCTC
CTGGTCCTAGGTGTTTCTAAGAAGCCATCACTCTCAGTGCAGCCAGGTCCT
ATCGTGGCCCCTGAGGAGACCCTGACTCTGCAGTGTGGCTCTGATGCTGGC
TACAACAGATTTGTTCTGTATAAGGACGGGGAACGTGACTTCCTTCAGCTC
GCTGGCGCACAGCCCCAGGCTGGGCTCTCCCAGGCCAACTTCACCCTGGGC
CCTGTGAGCCGCTCCTACGGGGGCCAGTACAGATGCTACGGTGCACACAAC
CTCTCCTCCGAGTGGTCGGCCCCCAGCGACCCCCTGGACATCCTGATCGCA
GGACAGTTCTATGACAGAGTCTCCCTCTCGGTGCAGCCGGGCCCCACGGTG
GCCTCAGGAGAGAACGTGACCCTGCTGTGTCAGTCACAGGGATGGATGCAA
ACTTTCCTTCTGACCAAGGAGGGGGCAGCTGATGACCCATGGCGTCTAAGA
TCAACGTACCAATCTCAAAAATACCAGGCTGAATTCCCCATGGGTCCTGTG
ACCTCAGCCCATGCGGGGACCTACAGGTGCTACGGCTCACAGAGCTCCAAA
CCCTACCTGCTGACTCACCCCAGTGACCCCCTGGAGCTCGTGGTCTCAGGA
CCGTCTGGGGGCCCCAGCTCCCCGACAACAGGCCCCACCTCCACATCTGCA
GGCCCTGAGGACCAGCCCCTCACCCCCACCGGGTCGGATCCCCAGAGTGGT
CTGGGAAGGCACCTGGGGGTTGTGATCGGCATCTTGGTGGCCGTCATCCTA
CTGCTCCTCCTCCTCCTCCTCCTCTTCCTCATCCTCCGACATCGACGTCAG
GGCAAACACTGGACATCGACCCAGAGAAAGGCTGATTTCCAACATCCTGCA
GGGGCTGTGGGGCCAGAGCCCACAGACAGAGGCCTGCAGTGGAGGTCCAGC
CCAGCTGCCGATGCCCAGGAAGAAAACCTCTATGCTGCCGTGAAGCACACA
CAGCCTGAGGATGGGGTGGAGATGGACACTCGGCAGAGCCCACACGATGAA
GACCCCCAGGCAGTGACGTATGCCGAGGTGAAACACTCCAGACCTAGGAGA
GAAATGGCCTCTCCTCCTTCCCCACTGTCTGGGGAATTCCTGGACACAAAG
GACAGACAGGCGGAAGAGGACAGGCAGATGGACACTGAGGCTGCTGCATCT
GAAGCCCCCCAGGATGTGACCTACGCCCAGCTGCACAGCTTGACCCTCAGA
CGGGAGGCAACTGAGCCTCCTCCATCCCAGGAAGGGCCCTCTCCAGCTGTG
CCCAGCATCTACGCCACTCTGGCCATCCACTAGCCCAGGGGGGGACGCAGA
CCCCACACTCCATGGAGTCTGGAATGCATGGGAGCTGCCCCCCCAGTGGAC
ACCATTGGACCCCACCCAGCCTGGATCTACCCCAGGAGACTCTGGGAACTT
TTAGGGGTCACTCAATTCTGCAGTATAAATAACTAATGTCTCTACAATTTT
GAAATAAAGCAACAGACTTCTCAATAATCAATGAAGTAGCTGAGAAAACTA
AGTCAGAAAGTGCATTAAACTGAATCACAATGTAAATATTACACATCAAGC
GATGAAACTGGAAAACTACAAGCCACGAATGAATGAATTAGGAAAGAAAAA
AAGTAGGAAATGAATGATCTTGGCTTTCCTATAAGAAATTTAGGGCAGGGC
ACGGTGGCTCACGCCTGTAATTCCAGCACTTTGGGAGGCCGAGGCGGGCAG
ATCACGAGTTCAGGAGATCGAGACCATCTTGGCCAACATGGTGAAACCCTG
TCTCTCCTAAAAATACAAAAATTAGCTGGATGTGGTGGCAGTGCCTGTAAT
CCCAGCTATTTGGGAGGCTGAGGCAGGAGAATCGCTTGAACCAGGGAGTCA
GAGGTTTCAGTGAGCCAAGATCGCACCACTGCTCTCCAGCCTGGCGACAGA
GGGAGACTCCATCTCAAATTAAAAAAAAAAAAAAAAAAGAAAGAAAAAAAA
AAAAAAAAAA
Human LILRB1 protein
(SEQ ID NO: 35)
MTPILTVLICLGLSLGPRTHVQAGHLPKPTLWAEPGSVITQGSPVTLRC
QGGQETQEYRLYREKKTAPWITRIPQELVKKGQFPIPSITWEHTGRYRC
YYGSDTAGRSESSDPLELVVTGAYIKPTLSAQPSPVVNSGGNVTLQCDS
QVAFDGFILCKEGEDEHPQCLNSQPHARGSSRAIFSVGPVSPSRRWWYR
CYAYDSNSPYEWSLPSDLLELLVLGVSKKPSLSVQPGPIVAPEETLTLQ
CGSDAGYNRFVLYKDGERDFLQLAGAQPQAGLSQANFTLGPVSRSYGGQ
YRCYGAHNLSSEWSAPSDPLDILIAGQFYDRVSLSVQPGPTVASGENVT
LLCQSQGWMQTFLLTKEGAADDPWRLRSTYQSQKYQAEFPMGPVTSAHA
GTYRCYGSQSSKPYLLTHPSDPLELVVSGPSGGPSSPTTGPTSTSAGPE
DQPLTPTGSDPQSGLGRHLGVVIGILVAVILLLLLLLLLFLILRHRRQG
KHWTSTQRKADFQHPAGAVGPEPTDRGLQWRSSPAADAQEENLYAAVKH
TQPEDGVEMDTRQSPHDEDPQAVTYAEVKHSRPRREMASPPSPLSGEFL
DTKDRQAEEDRQMDTEAAASEAPQDVTYAQLHSLTLRREATEPPPSQEG
PSPAVPSIYATLAIH

In the present invention, the binding inhibitor is only required to inhibit the binding between a RIFIN protein and a LILRB1 protein. The inhibition may be, for example, direct inhibition or indirect inhibition. The inhibition means, for example, that the formation amount of the complex of a RIFIN protein and a LILRB1 protein when the complex is formed in the presence of the binding inhibitor is (e.g., significantly) reduced compared to the formation amount of the complex when the complex is formed in the absence of the binding inhibitor, for example. The formation of the complex of a RIFIN protein and a LILRB1 protein can be measured, for example, with reference to from Examples described below, by bringing a labeled RIFIN protein or LILRB1 protein into contact with a subject expressing a LILRB1 protein or a RIFIN protein, and detecting the label in the subject after the contact. The measurement can be performed using, for example, a flow cytometer or the like.

The binding inhibitor may be a binding substance that binds to the RIFIN protein, a binding substance that binds to the LILRB1 protein, or the like. When the binding inhibitor is a binding substance that binds to the RIFIN protein, the binding inhibitor is preferably a binding substance that binds to a variable region of the RIFIN protein. The binding substance that binds to the variable region of the RIFIN protein binds to a part or all of the variable region of the RIFIN protein, for example. The variable and conserved regions of the RIFIN protein mean the amino acid regions described below, for example.

(Variable and Conserved Regions of RIFIN Protein)
PF3D7_0100200 (SEQ ID NO: 1, GenBank: CAB89210.1)
Variable region: an amino acid region extending from 164to to 291st amino acid residues in the amino acid sequence of SEQ ID NO: 1
Conserved region: an amino acid region extending from 43rd to 144th amino acid residues in the amino acid sequence of SEQ ID NO: 1
PF3D7_0900200 (SEQ ID NO: 2, GenBank: CAD51688.1)
Variable region: an amino acid region extending from 167th to 333rd amino acid residues in the amino acid sequence of SEQ ID NO: 2
Conserved region: an amino acid region extending from 42nd to 143rd amino acid residues in the amino acid sequence of SEQ ID NO: 2
PF3D7_0223100 (SEQ ID NO: 3, GenBank: CZT98243.1)
Variable region: an amino acid region extending from 162nd to 288th amino acid residues in the amino acid sequence of SEQ ID NO: 3
Conserved region: an amino acid region extending from 39th to 136th amino acid residues in the amino acid sequence of SEQ ID NO: 3
PF3D7_1254800 (SEQ ID NO: 4, GenBank: CZT99701.1)
Variable region: an amino acid region extending from 166th to 275th amino acid residues in the amino acid sequence of SEQ ID NO: 4
Conserved region: an amino acid region extending from 39th to 139th amino acid residues in the amino acid sequence of SEQ ID NO: 4
PF3D7_0700200 (SEQ ID NO: 5, GenBank: CZT62652.1)
Variable region: an amino acid region extending from 167th to 281st amino acid residues in the amino acid sequence of SEQ ID NO: 5
Conserved region: an amino acid region extending from 45th to 146th amino acid residues in the amino acid sequence of SEQ ID NO: 5
PF3D7_1100400 (SEQ ID NO: 6, GenBank: CZT98672.1)
Variable region: an amino acid region extending from 175th to 281st amino acid residues in the amino acid sequence of SEQ ID NO: 6
Conserved region: an amino acid region extending from 45th to 147th amino acid residues in the amino acid sequence of SEQ ID NO: 6
PF3D7_1480000 (SEQ ID NO: 7, GenBank: CZU00495.1)
Variable region: an amino acid region extending from 167th to 300th amino acid residues in the amino acid sequence of SEQ ID NO: 7
Conserved region: an amino acid region extending from 38th to 142nd amino acid residues in the amino acid sequence of SEQ ID NO: 7
PF3D7_0600300 (SEQ ID NO: 8, GenBank: CAG25174.1)
Variable region: an amino acid region extending from 166th to 329th amino acid residues in the amino acid sequence of SEQ ID NO: 8
Conserved region: an amino acid region extending from 40th to 145th amino acid residues in the amino acid sequence of SEQ ID NO: 8
PF3D7_0632700 (SEQ ID NO: 9, GenBank: CAG25139.1)
Variable Region: an amino acid region extending from 165th to 334th amino acid residues in the amino acid sequence of SEQ ID NO: 9
Conserved region: an amino acid region extending from 39th to 144th amino acid residues in the amino acid sequence of SEQ ID NO: 9
PF3D7_0632200 (SEQ ID NO: 10, GenBank: CAG25134.1)
Variable region: an amino acid region extending from 168th to 334th amino acid residues in the amino acid sequence of SEQ ID NO: 10
Conserved region: an amino acid region extending from 42nd to 143rd amino acid residues in the amino acid sequence of SEQ ID NO: 10
PF3D7_0100400 (SEQ ID NO: 11, GenBank: CAB89212.1)
Variable region: an amino acid region extending from 164th to 329th amino acid residues in the amino acid sequence of SEQ ID NO: 11
Conserved region: an amino acid region extending from 39th to 146th amino acid residues in the amino acid sequence of SEQ ID NO: 11
PF3D7_1254400 (SEQ ID NO: 12, GenBank: CZT99697.1)
Variable region: an amino acid region extending from 169th to 336th amino acid residues in the amino acid sequence of SEQ ID NO: 12
Conserved region: an amino acid region extending from 44th to 148th amino acid residues in the amino acid sequence of SEQ ID NO: 12

The type of the binding inhibitor is not particularly limited, and examples thereof include low molecular weight compounds, peptides, proteins, and nucleic acids. As a specific example, when the binding inhibitor is a peptide or a protein, the binding inhibitor may be, for example, an antibody or an antigen binding fragment thereof. The antibody may be, for example, a monoclonal antibody or a polyclonal antibody (hereinafter, the same applies). When the binding inhibitor is a peptide or a protein, the binding inhibitor may be, for example, a protein or a peptide that binds to a RIFIN protein, a protein or a peptide that binds to a LILRB1 protein, or the like. The protein or peptide that binds to a RIFIN protein may be, for example, an antibody that binds to a RIFIN protein or an antigen-binding fragment thereof, a solubilized product of a LILRB1 protein or a RIFIN protein-binding fragment thereof, or the like. The solubilized product of a LILRB1 protein may be, for example, a fusion protein of the Fc-region (fragment crystallizable region) of the antibody and a LILRB1 protein or a RIFIN protein-binding fragment thereof. The protein or peptide that binds to the LILRB1 protein may be, for example, an antibody that binds to a LILRB1 protein or an antigen-binding fragment thereof, a decoy peptide of a RIFIN protein, or the like. The decoy peptide of a RIFIN protein is, for example, a peptide that inhibits the binding of a RIFIN protein to a LILRB1 protein and that does not activate a LILRB1 protein. The activation of a LILRB1 protein means, for example, that the function of the LILRB1 protein is exerted. The peptide may be, for example, a cyclic peptide or a special cyclic peptide (hereinafter, the same applies). When the binding inhibitor is a nucleic acid, the binding inhibitor may be, for example, a binding nucleic acid molecule that binds to a RIFIN protein, a binding nucleic acid molecule that binds to a LILRB1 protein, or the like. The binding nucleic acid molecule may be, for example, DNA, RNA, or an aptamer consisting of DNA and RNA. For example, one type of the binding inhibitors may be used alone, or two or more types of them may be used in combination.

The inducer of the binding inhibitor is, when administered to a patient (hereinafter, also referred to as an “administration subject”), a substance that induces the binding inhibitor in the administration subject, for example. The binding inhibitor is preferably an antibody. Examples of the type of the inducer include a peptide, a protein, and a nucleic acid. The inducer is preferably used in combination with the adjuvant to be described below, for example, so that the binding inhibitor can be efficiently induced. The inducer may be, for example, the RIFIN protein or a part of the RIFIN protein, the LILRB1 protein or a part of the LILRB1 protein, or a polynucleotide encoding at least one of them. The part of the RIFIN protein preferably includes a variable region of a RIFIN protein, for example, so that a binding inhibitor that effectively inhibits the binding between a RIFIN protein and a LILRB1 protein can be induced. In this case, the inducer includes, for example, a part or all of the variable region of a RIFIN protein. When the inducer is the polynucleotide, the polynucleotide is functionally linked to, for example, a vector. The vector may be, for example, a known vector such as an adenovirus vector. The polynucleotide may be, for example, a DNA consisting of a deoxyribonucleotide, an RNA consisting of a ribonucleotide, or a polynucleotide consisting of DNA and RNA. The inducer may induce the expression of the binding inhibitor in the administration subject, for example. In this case, examples of the inducer include a polynucleotide encoding a synthetase of a low molecular weight compound, a peptide, or a protein; a polynucleotide containing a nucleic acid serving as a template of the nucleic acid serving as the binding inhibitor; and a vector containing the same. For example, one type of the inducers may be used alone or two or more types of them may be used in combination.

The expression inhibitor is not particularly limited, and examples thereof include a substance that inhibits an expression of mRNA of a RIFIN gene or a LILRB1 gene; a substance that cleaves expressed mRNA, and a substance that inhibits translation of a protein from the expressed mRNA. Specific examples of the expression inhibitor include a RNA-interfering agent such as siRNA, an antisense, and a ribozyme. The expression inhibitor may be, for example, a gene editing system capable of editing the base sequence of at least one of a RIFIN gene of Plasmodium falciparum and a LILRB1 gene of the patient. The genetic editing system may be, for example, a known gene editing system such as ZFN, TALEN, CRISPR-Cas9, or the like or a known mRNA editing system such as CRISPR-Cas13, or the like. For example, one type of the expression inhibitors may be used alone or two or more types of them may be used in combination.

The antimalarial drug of the present invention is only required to include any one or more of the binding inhibitor, the inducer, and the expression inhibitor, and may include two or all of them.

When the type of the antimalarial drug of the present invention is a peptide, a protein, or a nucleic acid, the peptide, the protein, and the nucleic acid may be modified and/or varied with one or more amino acids or one or more nucleotides, for example.

The modification is not particularly limited, and may be, for example, modification of the N-terminal, the C-terminal, or the side chain of a peptide or a protein, modification of a base, a sugar, a phosphate group, or a sugar phosphate backbone of a nucleic acid, or the like.

When the antimalarial drug of the present invention is a peptide or a protein, the variant of the peptide or the protein may be, for example, a peptide or a protein consisting of an amino acid sequence in which one to several amino acids are deleted, substituted, inserted and/or added in the amino acid sequence of the peptide or the protein, and/or a peptide or a protein consisting of an amino acid sequence having 80% or more identity with respect to the amino acid sequence of the peptide or protein. When the peptide or protein is a RIFIN protein or a part thereof, the variant binds to a LILRB1 protein, for example. Also, when the peptide or the protein is a LILRB1 protein or a part thereof, the variant binds to a RIFIN protein, for example. When the peptide or the protein is an antibody that binds to a RIFIN protein or an antigen-binding fragment thereof, the variant binds to a LILRB1 protein, for example. When the peptide or the protein is an antibody that binds to a LILRB1 protein or an antigen-binding fragment thereof, the variant binds to a RIFIN protein, for example. The foregoing numerical range of “one to several” include, for example, 1 to 76, 1 to 56, 1 to 37, 1 to 18, 1 to 15, 1 to 11, 1 to 7, 1 to 3, or 1 or 2. In the present invention, for example, the numerical range regarding the number of amino acids or the like discloses all the positive integers falling within that range. That is, for example, the numerical range of “1 to 5” discloses all of “1, 2, 3, 4, and 5” (the same applies hereinafter). The “identity” is, for example, 80% or more, 85% or more, 90% or more, 95% or more, 96% or more, 97% or more, 98% or more, or 99% or more. The “identity” can be calculated by default parameters using analysis software such as BLAST, FASTA, or the like (hereinafter, the same applies).

When the antimalarial drug of the present invention is a nucleic acid, the variant of the nucleic acid may be, for example, a polynucleotide consisting of a base sequence in which one to several bases are deleted, substituted, inserted and/or added in the base sequence of the nucleic acid, and/or a polynucleotide consisting of a base sequence having 80% or more identity with respect to the base sequence of the nucleic acid. When the nucleic acid is a nucleic acid encoding a RIFIN protein or a part thereof, the protein or the peptide encoded by the variant binds to a LILRB1 protein, for example. Also, when the nucleic acid is a nucleic acid encoding a LILRB1 protein or a part thereof, the protein or the peptide encoded by the variant binds to a RIFIN protein, for example. When the nucleic acid is a nucleic acid that binds to a RIFIN protein, the variant binds to a LILRB1 protein, for example. When the nucleic acid is a nucleic acid that binds to a LILRB1 protein, the variant binds to a RIFIN protein, for example. The foregoing numerical range of “one to several” include, for example, 1 to 228, 1 to 168, 1 to 111, 1 to 54, 1 to 45, 1 to 33, 1 to 21, 1 to 9, 1 to 6, 1 to 3, or 1 or 2. The “identity” is, for example, 80% or more, 85% or more, 90% or more, 95% or more, 96% or more, 97% or more, 98% or more, or 99% or more.

The antimalarial drug of the present invention may contain other components, such as, for example, a pharmaceutically acceptable carrier. In this case, the antimalarial drug of the present invention can also be referred to as a “pharmaceutical composition”, for example. The other components are not particularly limited, and examples thereof include preservatives, antioxidants, chelating agents, stabilizing agents, emulsifying agents, dispersing agents, suspending agents, and thickening agents. Examples of the preservative include thimerosal and 2-phenoxyethanol. Examples of the chelating agent include an ethylenediaminetetraacetic acid and a glycol ether diaminetetraacetic acid.

When the antimalarial drug of the present invention includes the inducer, the antimalarial drug of the present invention preferably includes an adjuvant as described above. In this case, the antimalarial drug of the present invention can also be referred to, for example, as a “vaccine” or a “vaccine composition”. The adjuvant is not particularly limited, and examples thereof include known adjuvants such as aluminum hydroxide, aluminum phosphate, aluminum chloride, lipopolysaccharide (LPS), Poly (I:C) (Polyinosinic-polycytidylic acid), complete Freund's adjuvant, incomplete Freund's adjuvant, Toll like receptor stimulators including CpG oligonucleotides, and the like, and saponins.

The antimalarial drug of the present invention can directly or indirectly inhibit the binding between a RIFIN protein and a LILRB1 protein, for example, by administrating on to an administration subject. As a specific example, the binding inhibitor inhibits, by binding to a RIFIN protein or a LILRB1 protein, binding of a LILRB1 protein to a RIFIN protein or a RIFIN protein to a LILRB1 protein, for example. The inducer induces the binding inhibitor, for example, in the administration subject, thereby inhibiting binding of a LILRB1 protein to a RIFIN protein or binding of a RIFIN protein to a LILRB1 protein. In addition, the expression inhibitor inhibits, by inhibiting expression of a RIFIN protein or a LILRB1 protein in the administration subject, binding of a LILRB1 protein to a RIFIN protein or binding of a RIFIN protein to a LILRB1 protein, for example.

For example, as described above, the antimalarial drug of the present invention can directly or indirectly inhibit the binding between a RIFIN protein and a LILRB1 protein, so that the generation of a signal through a LILRB1 protein by a RIFIN protein can be inhibited or the signal can be suppressed. Therefore, according to the antimalarial drug of the present invention, for example, the suppression of the function of the immune system cells by the binding between a RIFIN protein and a LILRB1 protein can be prevented or released. Thus, the antimalarial drug of the present invention may contain, for example, a signal inhibitor that inhibits a signal through a LILRB1 protein in addition to or in place of the binding inhibitor, the inducer, and/or the expression inhibitor. Examples of the signal inhibitor include an antagonist of LILRB1 protein, an active inhibitor of a signal transduction protein of LILRB1 protein, the binding inhibitor, the inducer, and the expression inhibitor. The type of the signal inhibitor is not particularly limited, and examples thereof include low molecular weight compounds, peptides, proteins, and nucleic acids. The inhibition of the signal means, for example, that the amount of a signal through a LILRB1 protein when the complex of a ligand (e.g., RIFIN protein) of a LILRB1 protein and a LILRB1 protein is formed in the presence of the signal inhibitor is (e.g., significantly) reduced compared to the amount of a signal through a LILRB1 protein in the absence of the signal inhibitor, for example. The amount of the signal can be measured, for example, with reference to the Examples described below, using reporter cells expressing reporter genes when a signal through a LILRB1 protein is generated. The measurement can be performed using, for example, a flow cytometer or the like.

In the present invention, examples of the administration subject (patient) include humans and non-human animals excluding humans. Examples of the non-human animals include mice, rats, dogs, monkeys, rabbits, sheep, horses, guinea pigs, and cats.

The dose of the antimalarial drug of the present invention is not particularly limited, and can be set as appropriate depending on the type of the antimalarial drug, the type, symptom, and age of the administration subject, and the administration method, for example. As a specific example, when the antimalarial drug of the present invention is a peptide and the antimalarial drug is administered to a human, the dose of the peptide per administration is 0.8 to 30 mg or 10 to 30 mg, for example. The number of administrations of the peptide is not particularly limited, and is 1 to 3 times, for example.

When the antimalarial drug of the present invention is a protein and the antimalarial drug is administered to a human, the dose of the protein per administration is 1 to 100 mg or 10 to 1000 mg, for example. The number of administrations of the protein is not particularly limited, and is 1 to 5 times, for example.

The administration form (dosage form) of the antimalarial drug of the present invention is not particularly limited, and examples thereof include solutions, suspensions, emulsions, injections, sprays, and powders.

The administration method of the antimalarial drug of the present invention is not particularly limited, and may be, for example, intravenous injection, intramuscular injection, subcutaneous administration, intradermal administration, transdermal administration, rectal administration, intraperitoneal administration, local administration, transnasal administration, or sublingual administration.

The method for producing the antimalarial drug of the present invention is not particularly limited, and, for example, a known method can be adopted depending on the type of the antimalarial drug. When the antimalarial drug of the present invention includes a low-molecular compound, the low-molecular compound can be produced by a known method, for example, depending on its structure.

When the antimalarial drug of the present invention includes a peptide, examples of the method for producing the peptide include a chemical synthesis method, a method for producing the peptide by degradation of a protein containing the peptide, and a synthesis method using recombinant DNA technology. In the chemical synthesis method, the peptide can be produced by a known organic synthesis method using, for example, a protecting group such as a benzyloxycarbonyl group (Cbz), a tert-butoxycarbonyl group (Boc), a fluorenylmethoxycarbonyl group (Fmoc), or the like. When the peptide is produced by the degradation of the protein containing the peptide, the peptide can be produced, for example, by degrading the protein containing the peptide with a known proteolytic enzyme such as a protease, a peptidase, or the like. The degradation condition of the protein containing the peptide can be set as appropriate depending on, for example, the type of the protein containing the peptide, the substrate specificity of the proteolytic enzyme, and the like. When the peptide is produced using the recombinant DNA technology, for example, an expression vector containing a polynucleotide encoding the peptide, is created, then, an expression system of the peptide is produced, and the expressed peptide is isolated, thereby producing the peptide. The expression system can be produced, for example, by introducing the expression vector into a host. Examples of the host include known hosts such as animal cells, plant cells, insect cells, and bacteria. When the peptide is produced using the recombinant DNA technology, for example, a polynucleotide encoding the peptide and a known cell-free translation system may be used. The peptide can be produced by isolating the peptide translated from the polynucleotide by the cell-free translation system.

When the antimalarial drug of the present invention includes a protein, examples of the method for producing the protein include a chemical synthesis method and a synthesis method using recombinant DNA technology. Regarding the method for producing the protein, for example, reference can be made to the description as to the method for producing the peptide by replacing “peptide” with “protein”. When the protein contains an antibody, the method for producing the antibody may be, for example, the same as the method for producing the protein, a method for immunizing the animal with an antigen of the antibody and then collecting a serum, or a method for culturing cells such as hybridomas that produce the antibody.

When the antimalarial drug of the present invention includes a nucleic acid, the method for producing the nucleic acid is not particularly limited, and examples thereof include a chemical synthesis method such as a phosphoramidite method and a synthesis method using recombinant DNA technology. When the nucleic acid contains an aptamer, the aptamer can be obtained by the SELEX method using the RIFIN protein or the LILRB1 protein, for example.

<Malaria Treatment Method>

The malaria treatment method of the present invention is characterized in that the method includes administering the antimalarial drug of the present invention to a patient, as described above. The treatment method of the present invention is characterized in that the method includes administering the antimalarial drug of the present invention to a patient, and the other steps and conditions are not particularly limited. According to the treatment method of the present invention, it is possible to treat malaria such as preventing malaria from becoming severe. Regarding the treatment method of the present invention, for example, reference can be made to the description as to the antimalarial drug of the present invention.

The treatment method of the present invention may be performed, for example, on a patient who is determined to be at risk of malaria becoming severe in the test method of the present invention to be described below. In this case, the treatment method of the present invention may be carried out, for example, in combination with the test method of the present invention. Regarding the specific steps of testing the risk of severe malaria, reference can be made to the description as to the test method of the present invention. The patient may be a patient not infected with malaria, a patient infected with malaria, or a patient with unknown malaria status.

<Screening Method for Candidate Substance for Malaria Treatment>

As described above, the method for screening a candidate substance for malaria treatment of the present invention includes: selecting, as a candidate substance for malaria treatment, a binding inhibitor that inhibits binding between a RIFIN protein and a leukocyte immunoglobulin-like receptor subfamily B member 1 (LILRB1) protein, an inducer of the binding inhibitor; or an expression inhibitor of RIFIN or LILRB1 from a test substance. The screening method of the present invention is characterized in that the candidate substance for malaria treatment is a binding inhibitor that inhibits binding between a RIFIN protein and a LILRB1 protein, an inducer of the binding inhibitor; or an expression inhibitor of RIFIN or LILRB1, and other steps and condition are not particularly limited. Regarding the screening method of the present invention, reference can be made to the description as to the antimalarial drug and treatment method of the present invention, for example.

Examples of the type of the binding substance include a low molecular weight compound, a peptide, a protein, and a nucleic acid. The RIFIN is preferably a LILRB1-binding RIFIN so that it can effectively treat malaria, for example.

The screening method of the present invention includes: detecting binding between the RIFIN protein and the LILRB1 protein in the presence of the RIFIN protein, the LILRB1 protein, and the test substance; and selecting, as the candidate substance for malaria treatment, the test substance that inhibits binding between the RIFIN protein and the LILRB1 protein, for example. In the detecting, the method of detecting the binding between a RIFIN protein and a LILRB1 protein is not particularly limited, and, for example, a known method of detecting the binding between proteins can be adopted, and reference can be made to the Examples to be described below.

The screening method of the present invention includes: administering the test substance to a living organism (administration subject); collecting a biological sample from the living organism; detecting binding between the RIFIN protein and the LILRB1 protein in the presence of the RIFIN protein, theLILRB1 protein, and the biological sample; and selecting, as the candidate substance for malaria treatment, the test substance that inhibits binding between the RIFIN protein and the LILRB1 protein, for example. In the administering, regarding the administration condition of the test substance, for example, reference can be made to the description as to the antimalarial drug of the present invention. The administering and the collecting are optional steps and may not be included. In such a case, in the detecting, a biological sample collected from a living organism to which the test substance has been administered is used as the biological sample, for example. The biological sample is not particularly limited, and examples thereof include blood, plasma, and serum.

The screening method of the present invention includes: bringing the test substance into contact with the RIFIN protein or the LILRB1 protein; detecting binding between the RIFIN protein or the LILRB1 protein and the test substance; and selecting, as the candidate substance for malaria treatment, the test substance binding to the RIFIN protein or the LILRB1 protein, for example.

The screening method of the present invention includes: causing the test substance to be coexist in an expression system of the RIFIN or the LILRB1 to express the RIFIN or the LILRB1; detecting an expression of the RIFIN or the LILRB1 in the expression system; and selecting, as the candidate substance for malaria treatment, the test substance with which the expression level of the RIFIN or the LILRB1 is lower than that of a control expression system in which the test substance is not present, for example. In detecting, the expression of the detection target may be the expression of the RIFIN protein or the LILRB1 protein, or the transcription of an mRNA of a RIFIN gene or a LILRB1 gene, for example. The methods for detecting the expression of the protein and the expression of the mRNA are not particularly limited, and known methods can be adopted, for example.

The screening method of the present invention may select, in addition to or in place of the binding inhibitor, the inducer, and/or the expression inhibitor, a signal inhibitor that inhibits a signal through a LILRB1 protein as a candidate substance for malaria treatment, for example. Examples of the signal inhibitor include an antagonist of LILRB1 protein, an active inhibitor of the signal transduction molecule such as a signal transduction protein of LILRB1 protein and the like, the binding inhibitor, the inducer, and the expression inhibitor. When the screening method of the present invention selects a signal inhibitor as the candidate substance for malaria treatment, the screening method of the present invention includes: detecting a signal through a LILRB1 protein in the presence of a ligand of the LILRB1 protein such as the RIFIN protein, the LILRB1 protein, and the test substance; and selecting the test substance that inhibits a signal through the LILRB1 protein as the candidate substance for malaria treatment, for example. In the detecting, the method for detecting a signal through the LILRB1 protein is not particularly limited, and, for example, a known detection method of signal transduction molecule can be adopted, and, for example, reporter cells in the Examples to be described below can be used.

<Malaria Severity Marker>

The malaria severity marker of the present invention is characterized in that the marker is RIFIN, as described above. The marker of the present invention is characterized in that RIFIN is used as the marker, and the other configurations and conditions are not particularly limited. The marker of the present invention can test the risk of severe malaria in the subject by measuring the expression of RIFIN in the biological sample of the subject, for example. Regarding the marker of the present invention, for example, reference can be made to the description as to the antimalarial drug, treatment method, and screening method of the present invention. The malaria is, for example, malaria caused by infection of the Plasmodium falciparum. The RIFIN is preferably a LILRB1-binding RIFIN because it is more correlated with the severe malaria, for example.

<Test Method>

The method for testing the risk of severe malaria of the present invention is characterized in that it includes measuring an expression of RIFIN in a biological sample of a subject, as described above. The test method of the present invention is characterized in that the expression of RIFIN is measured as a malaria severity marker, and other steps and conditions are not particularly limited. According to the test method of the present invention, it is possible to test the subject's risk of severe malaria. Regarding the test method of the present invention, for example, reference can be made to the description as to the antimalarial drug, treatment method, screening method, and marker of the present invention.

According to the test method of the present invention, for example, it is possible to evaluate the possibility of the progression of the malaria symptom, the possibility of malaria becoming severe, the evaluation of the prognosis, and the like. The malaria is, for example, malaria caused by infection of the Plasmodium falciparum.

Exampes of the subject include humans and non-human animals excluding humans. Examples of the non-human animals include, as described above, mammals such as mice, rats, dogs, monkeys, rabbits, sheep, horses, and the like.

The type of the biological sample is not particularly limited, and examples thereof include body fluids, body fluid-derived cells, organs, tissues, and cells separated from a living organism. The body fluid may be, for example, blood, and specifically, for example, whole blood. The body fluid-derived cells may be, for example, blood-derived cells, and specifically, blood cells such as hemocyte, white blood cells, erythrocytes, lymphocytes, and the like. Since RIFIN is expressed in, for example, erythrocytes in a patient infected with Plasmodium falciparum, the biological sample is preferably a biological sample containing blood or a biological sample containing erythrocytes.

The expression of RIFIN to be measured may be, for example, the expression of mRNA of a RIFIN gene or the expression of a RIFIN protein. With respect to the biological sample, only one of the expression of the mRNA and the expression of the protein or both of them may be measured. These measurement methods are not particularly limited, and known methods can be adopted. As a specific example, the method for measuring the expression of the mRNA may be, for example, a gene amplification method using a reverse transcription reaction such as a reverse transcription (RT)-PCR method. Specifically, for example, the cDNA is synthesized from mRNA by reverse transcription, and cDNA is used as a template to amplify genes. Examples of the method for measuring the expression of the protein include an immuno-antibody method, an ELISA method, a flow cytometry, and a Western blotting method. The expression of a RIFIN protein may also be measured, for example, using a LILRB1 protein. In this case, as a LILRB1 protein, for example, the LILRB1-Fc to be described below can be used. The RIFIN is preferably a LILRB1-binding RIFIN because it is more correlated with the severe malaria, for example.

In the measuring, for example, the presence or absence of the expression of RIFIN may be measured, or the expression level of RIFIN may be measured.

The test method of the present invention further includes testing the subject's risk of severe malaria based on the expression of RIFIN in the biological sample of the subject (hereinafter also referred to as “subject biological sample”), for example. When the measurement result in the measuring is the expression level of RIFIN, the testing includes testing the subject's risk of severe malaria by comparing the expression level of RIFIN in the biological sample of the subject with a reference value, for example. The reference value is not particularly limited, and may be the expression levels of RIFIN in healthy subjects, severe malaria patients, and malaria patients at different severities, for example. For prognostic evaluation, the reference value may be, for example, the expression level of RIFIN after treatment.

The reference value can be obtained, for example, by using a biological sample isolated from a healthy subject and/or a severe malaria patient (hereinafter also referred to as a “reference biological sample”), as described above. In the case of prognostic evaluation, for example, a reference biological sample isolated from the same subject after treatment may be used. The reference value may be measured at the same time as the subject biological sample of the subject, or may be measured in advance, for example. The latter case is preferable because, for example, it is unnecessary to obtain a reference value every time the subject biological sample of the subject is measured. Preferably, the subject biological sample of the subject and the reference biological sample are collected under the same conditions, and RIFIN is measured under the same conditions, for example.

In the testing, the method of evaluating the subject's risk of severe malaria is not particularly limited, and can be appropriately determined, for example, based on the measurement result obtained in the measuring. When the measurement result indicates the presence or absence of the expression of RIFIN, for example, the measurement can be performed as follows. As a specific example, when RIFIN is expressed in the subject biological sample of the subject, the subject can be evaluated as being at a risk of or at a high risk of severe malaria. When RIFIN is not expressed in the subject biological sample of the subject, the subject can be evaluated as having no risk or at a low risk of severe malaria. When the measurement result is the expression level of RIFIN, the evaluation method can be determined as appropriate depending on the type of the reference value, for example. As specific examples, when the expression level of RIFIN in the subject biological sample of the subject is significantly higher than the expression level of RIFIN in the reference biological sample of the healthy subject, when the expression level of RIFIN in the subject biological sample of the subject is the same as the expression level of RIFIN in the reference biological sample of the severe malaria patient (when there is no significant difference therebetween), and/or when the expression level of RIFIN in the subject biological sample of the subject is significantly higher than the expression level of RIFIN in the reference biological sample of the malaria patient, the subject can be evaluate as at a risk or at a high risk of severe malaria. On the other hand, when the expression level of RIFIN in the subject biological sample of the subject is the same as the expression level of RIFIN in the reference biological sample of the healthy subject (when there is no significant difference therebetween), when the expression level of RIFIN in the subject biological sample of the subject is significantly lower than the expression level of RIFIN in the reference biological sample of the healthy subject, and/or when the expression level of RIFIN in the subject biological sample of the subject is significantly lower than the expression level of RIFIN in the reference biological sample of the severe malaria patient, the subject can be evaluated as having no risk or at a low risk of malaria severe malaria. In the testing, the degree of progression of the severity of malaria can be evaluated by comparing the expression level of RIFIN in the subject biological sample of the subject with the expression levels of RIFIN in the reference biological samples of the malaria patients at different severities. Specifically, when the subject biological sample of the subject has an expression level equivalent to that of the reference biological sample of a certain severity (when there is no significant difference therebetween), for example, the subject can be evaluated as having a possibility of being at the certain severity.

In the testing, when the prognostic state is evaluated, for example, the evaluation may be made in the same manner as described above or the evaluation may be made using the expression level of RIFIN in a reference biological sample of the same subject after treatment as a reference value. As a specific example, when the expression level of RIFIN in the subject biological sample of the subject is significantly higher than the reference value, the subject can be evaluated as at a risk of relapse or aggravation (becoming severe) after the treatment. On the other hand, when the expression level of RIFIN in the subject biological sample of the subject is the same as the reference value (when there is no significant difference therebetween) and/or is significantly lower than the reference value, the subject can be evaluated as having no risk or a low risk of relapse after the treatment.

In the present invention, for example, the biological samples of the same subject may be collected over time, and the expression levels of RIFIN in the biological samples may be compared. Thereby, it is possible to judge that the possibility of becoming severe increases if the expression level increases over time, and it is possible to judge that the possibility of becoming severe decreases or that malaria has healed if the expression level decreases over time, for example.

<Test Reagent>

The test reagent of the present invention is characterized in that it includes a reagent for measuring an expression of RIFIN and is for use in the test method of the present invention, as described above. The test method of the present invention is characterized in that the reagent for measuring an expression of RIFIN is used in the test method of the present invention, and other configurations and conditions are not particularly limited. According to the test reagent of the present invention, the test method of the present invention can be easily carried out.

The expression measuring reagent may be any reagent as long as the expression of RIFIN can be measured, and the type of the expression measuring reagent is not particularly limited. The expression measurement reagent of RIFIN may be, for example, a reagent for measuring an expression of a RIFIN protein or a reagent for measuring an expression of mRNA of a RIFIN gene.

The former may be, for example, a binding substance that bind to a RIFIN protein, and specific examples thereof include an antibody and an antigen-binding fragment thereof. In this case, preferably, the test reagent of the present invention further contains, for example, a detection substance for detecting the binding between a RIFIN protein and the antibody or the antigen-binding fragment thereof. The detection substance can be, for example, a combination of a detectable labeled antibody to the antibody or the antigen-binding fragment thereof and a substrate to the label.

The latter may be, for example, a reagent that amplifies mRNA of a RIFIN gene by reverse transcription, and specific examples thereof include poly-dT, random primers, reverse transcriptase, dNTP, DNA polymerases, and primers. The primers can be designed as appropriate based on, for example, the base sequence of a RIFIN gene.

<Diagnostic Method and Diagnostic Reagent for Malaria Severity>

The method for diagnosing the severity of malaria is characterized in that it includes measuring the expression of RIFIN in a biological sample of a subject. The reagent for diagnosing the severity of malaria is characterized in that it includes a reagent for measuring the expression of RIFIN. Regarding the diagnostic method and diagnostic reagent of the present invention, for example, reference can be made to the description as to the test method and test reagent of the present invention.

<Use of Antimalarial Drug>

The present invention relates to a binding inhibitor that inhibits binding between a RIFIN protein and a leukocyte immunoglobulin-like receptor subfamily B member 1 (LILRB1 protein), an inducer of the binding inhibitor, or an expression inhibitor of RIFIN or LILRB1 for use in malaria treatment. The present invention relates to use of a binding inhibitor that inhibits binding between a RIFIN protein and a leukocyte immunoglobulin-like receptor subfamily B member 1 (LILRB1 protein), an inducer of the binding inhibitor, or an expression inhibitor of RIFIN or LILRB1 for producing an antimalarial drug. Regarding the use of the antimalarial drug of the present invention, for example, reference can be made to the description as to the antimalarial drug, treatment method, screening method, marker, test method, and test reagent of the present invention.

The present invention is described below in detail with reference to examples and the like. The present invention, however, is not limited thereto.

### EXAMPLES

### Example 1

The present example examined whether LILRB1 binds to infected erythrocytes infected with Plasmodium falciparum.

(1) Preparation of LILRB1-Fc

A plasmid encoding a LILRB1-Fc fusion protein (LILRB1-Fc expression vector) was prepared in the same manner as described in Reference 1 below. In order to produce a biotinylated LILRB1-Fc protein, a LILRB1-Fc expression vector having an AviTag added to the C-terminal (Avi-LILRB1-Fc expression vector) was produced by inserting a polynucleotide encoding LILRB1-Fc into a pCAGGS expression vector. 293T cells (purchased from RIKEN Cell Bank) were transfected with the LILRB1-Fc and Avi-LILRB1-Fc expression vectors using a transfection reagent (PEI Max, Polysciences Inc.) according to the attached protocols. Then a culture supernatant containing LILRB1-Fc and Avi-LILRB1-Fc was obtained by culturing the 293T cells. Protein A was used to purify LILRB1-Fc and Avi-LILRB1-Fc from the resulting culture supernatant.
Reference 1: Hirayasu, K. et al., “Microbially cleaved immunoglobulins are sensed by the innate immune receptor LILRA2.”, Nat. Microbiol., 2016, vol. 1, Article number:16054

(2) Preparation of Clinical Strains of Plasmodium falciparum

Clinical strains of Plasmodium falciparum (hereinafter also referred to as “protozoa”) were prepared by separate on from malaria patients (Patients 1 to 7) resident in Mae Sariang district, Mae Hong Son Province, Thailand, and subjecting to limiting dilution. The clinical strains were cultured in a RPMI-1640 medium (containing 20% AlbuMAXI™, 25 mmol/l HEPES, 0.225% sodium bicarbonate, 0.38 mmol/1 hypoxatine, and 10 g/ml gentamycin) supplemented with human erythrocytes (purchased from the Japanese Red Cross Society, type O, hematocrit 2%). The culture conditions were set under 90% N₂, 5% CO₂ and 5% O₂ atmosphere and at the culture temperature of 37° C.

(3) Culture of Plasmodium falciparum

The protozoa strains 3D7, CDC1, K1, FCR3 and Dd2, and the above-mentioned clinical strains were cultured in a RPMI-1640 medium with 10% human serum that contains human erythrocytes. The recombinant protozoa to be described below were maintained in a RPMI-1640 medium with 10% human serum and 25 ng/ml pyrimethamine (SIGMA). Using 5% D-sorbitol, the protozoa were synchronized to the ring stage. The protozoa in the trophozoite stage and the protozoa in the schizont stage were prepared by the Percoll density gradient centrifugation (GE Healthcare). Each Plasmodium falciparum was examined periodically for mycoplasma contamination by PCR.

(4) Binding Between LILRB1-Fc and Infected Erythrocyte

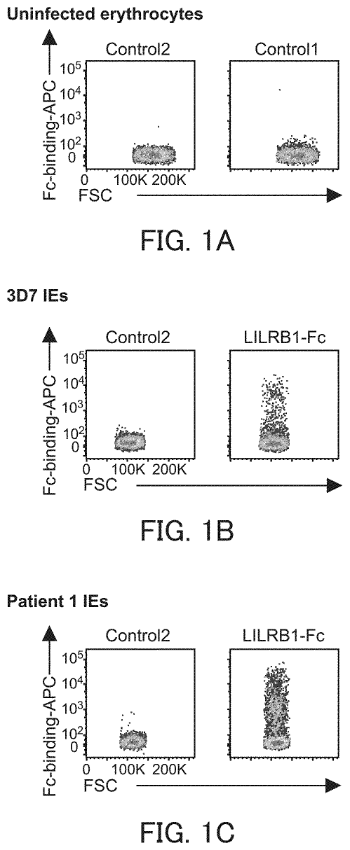

LILRB1-Fc was mixed with an APC-labeled anti IgG Fc antibody to form a complex. The complex was then incubated with infected erythrocytes infected with a 3D7 strain or with the clinical strain (derived from Patient 1) to stain the infected erythrocytes with the complex. After the staining, the resulting samples were analyzed by flow cytometry. As Control 1, the analysis was performed in the same manner except that the erythrocytes were not infected with protozoa, and as Control 2, the analysis was performed in the same manner except that only an APC-labeled anti IgG Fc antibody was used. FIG. 1 shows these results.

FIG. 1 shows dot plots illustrating the results of flow cytometry. In FIG. 1, (A) shows the results of the erythrocytes not infected with protozoa, (B) shows the results of the infected erythrocytes infected with 3D7 strain, and (C) shows the results of the infected erythrocytes infected with the clinical strain. In each of (A) to (C) of FIG. 1, the horizontal axis indicates the forward scattered light (F SC), and the vertical axis indicates the binding amount of LILRB1-Fc (Fc-binding-APC). As shown in (A) to (C) of FIG. 1, no binding of LILRB1-Fc was observed in Controls 1 and 2. In contrast, binding of LILRB1-Fc to the infected erythrocytes infected with either the 3D7 strain or the clinical strain was observed in the present example (LILRB1-Fc). These results showed that LILRB1-Fc binds to the infected erythrocytes infected with Plasmodium falciparum.

### Example 2

The present example examined whether a LILRB1 protein binds to infected erythrocytes infected with a clinical strain of Plasmodium falciparum.

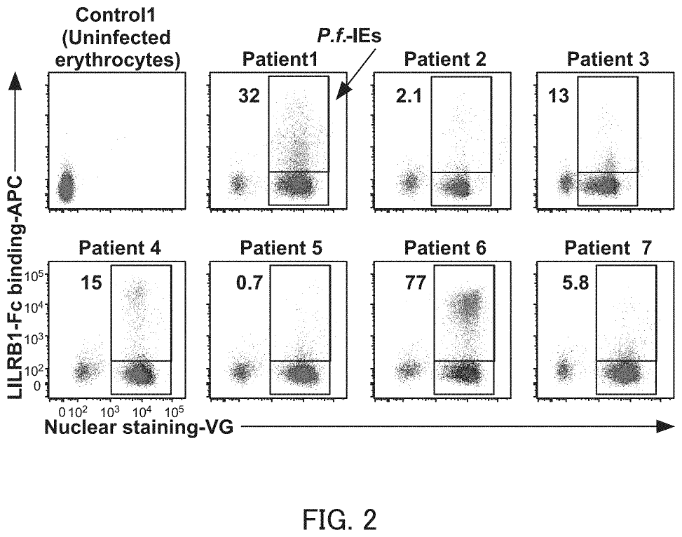

Infected erythrocytes were stained with a complex of LILRB1-Fc and an APC-labeled anti IgG Fc antibody in the same manner as in Example 1, using the clinical strains of protozoa separated from Patients 1 to 7. The infected erythrocytes were subjected to nuclear staining using a nuclear staining agent (Vybrant® DyeCycle™ Green). After the staining, the resulting samples were analyzed by flow cytometry. As Control 1, the analysis was performed in the same manner except that the erythrocytes were not infected with the clinical strain, and as Control 2, the analysis was performed in the same manner except that a LILRA2-Fc fusion protein was added in place of LILRB1-Fc. LILRA2-Fc was prepared in the same manner as described in Reference 1. FIG. 2 shows these results.

FIG. 2 shows dot plots illustrating the results of flow cytometry. In FIG. 2, the horizontal axis indicates the fluorescent intensity of the nuclear staining agent (Nuclear staining-VG), and the vertical axis indicates the binding amount of LILRB1-Fc (LILRB1-Fc binding-APC). The numerical values in FIG. 2 indicate the percentage of erythrocytes that are nuclear staining agent-positive in the present example and to which LILRB1-Fc binds. As shown in FIG. 2, no binding of LILRB1-Fc to the infected erythrocytes was observed in Control 1 (uninfected erythrocytes) and Control 2 (black plots) in the nuclear staining agent-positive group showing the infection with the clinical isolate. In contrast, LILRB1-Fc was efficiently bound to the infected erythrocytes infected with clinical strains derived from from Patients 1, 3, 4 and 6 in the present example (gray plots). No binding of LILRB1-Fc to the infected erythrocytes infected with protozoa derived from Patients 2 and 5 was observed. It is presumed that this is because the protozoa change the antigen expressed on the surface of the infected erythrocytes depending on the state of the host. This presumption, however, does not limit the present invention in any way. These results showed that a LILRB1 protein binds to the infected erythrocytes infected with a clinical strain of Plasmodium falciparum.

### Example 3

The present example examined whether a LILRB1 protein binds to infected erythrocytes infected with different types of Plasmodium falciparum or at different stages.

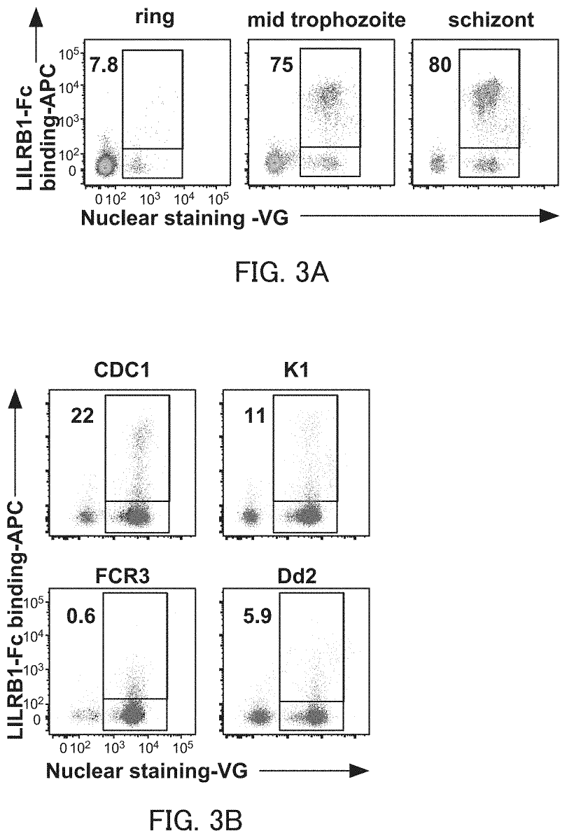

Erythrocytes infected with protozoa synchronized to the ring stage, infected erythrocytes infected with protozoa in the trophozoite stage, and infected erythrocytes infected with protozoa in the schizont stage were prepared by the method described in Example 1(3). As the protozoa, protozoa derived from Patient 6 were used. The analysis was performed in the same manner as in Example 2 except that the infected erythrocytes infected with protozoa in the ring stage, trophozoite stage, or schizont stage were used in place of the clinical strain. Furthermore, the analysis was performed in the same manner except that the CDC1 strain, K1 strain, FCR3 strain, and Dd2 strain were used in place of protozoa at different stages. In addition, as a control, the analysis was performed in the same manner except that LILRA2-Fc was added in place of LILRB1-Fc. FIG. 3 shows these results.

FIG. 3 shows dot plots illustrating the results of flow cytometry. In FIG. 3, the horizontal axis indicates the fluorescent intensity of the nuclear staining agent (Nuclear staining-VG), and the vertical axis indicates the binding amount of LILRB1-Fc (LILRB1-Fc binding-APC). In FIG. 3, (A) shows the results of the infected erythrocytes infected with protozoa at different stages, and (B) shows the results of infected erythrocytes infected with different types of protozoa. In (A) of FIG. 3, the dot plots show, from the left, the results of the infected erythrocytes infected with protozoa in the ring stage (ring), the results of the infected erythrocytes infected with protozoa in the trophozoite stage (mid trophozoite), and the results of the infected erythrocytes infected with protozoa in the schizont stage (schizont). In (B) of FIG. 3, the name on each dot plot indicates the name of the protozoa strain. The numerical values in FIG. 3 indicate the percentage of erythrocytes that are nuclear staining agent-positive in the present example and to which LILRB1-Fc binds. As shown in (A) of FIG. 3, LILRB1-Fc was bound to the infected erythrocytes infected with protozoa at any stage. LILRB1-Fc was efficiently bound to the infected erythrocytes infected with protozoa in particular in the trophozoite stage or schizont stage. As shown in (B) of FIG. 3, no binding of LILRB1-Fc to the infected erythrocytes was observed in the control (black plots) in the nuclear staining agent-positive group showing the infection with protozoa. In contrast, binding of LILRB1-Fc to the infected erythrocytes infected with protozoa of either CDC1 strain, K1 strain, and Dd2 strain was observed in the present example (grey plots). No binding of LILRB1-Fc to the infected erythrocytes infected with the FCR3 strain was observed. It is presumed that this is because the protozoa change the antigen expressed on the surface of the infected erythrocytes depending on the state of the host. These results showed that a LILRB1 protein binds to the infected erythrocytes infected with different types of Plasmodium falciparum or at different stages. The LILRB1 protein is known to recognize MHC-class 1 molecules. However, the erythrocytes used in Examples 1 to 3 do not express MHC-class 1 molecules. This showed that LILRB1-Fc binds to erythrocytes through molecules derived from Plasmodium falciparum expressed on erythrocytes as ligands.

### Example 4

The present example examined whether a ligand of a LILRB1 protein is a RIFIN protein. The present example also examined that a LILRB1 protein binds to various RIFIN proteins.

(1) Cloning Using Plasmodium falciparum 3D7 Strains

An APC-labeled anti IgG-Fc antibody was mixed with the LILRB1-Fc fusion protein to be described below to form a complex. The complex was then bonded to infected erythrocytes infected with a 3D7 strain. Then, the infected erythrocytes binding to LILRB1-Fc were enriched. The infected erythrocytes after enrichment were subjected to limiting dilution to obtain monoclonal protozoa. Specifically, the protozoa in the enriched infected erythrocytes were synchronized to the ring stage by culturing in the presence of 5% D-sorbitol. After replacing with a fresh medium and culturing for an additional 48 hours, the protozoa in the late schizont stage were purified by 63 (v/v)% Percoll (manufactured by Amersham Pharmacia Biotech) density gradient centrifugation method. The purified protozoa were mixed with a culture solution (RPMI-1640 medium with 10% O-type human serum, parasitemia: 1%, hematocrit: 2%) and cultured in a T-75 flask. The culture was carried out using a BNP-110 incubator (manufactured by TABAI ESPEC) for 1 hour at 37° C. under 90% N₂, 5% CO₂, and 5% O₂ atmosphere. After the culture, the cap of the flask was tightly closed, and the flask was shaken at 100 rpm using an orbital shaker so as to prevent one cell of erythrocytes from being infected with multiple protozoa. The stage of the protozoa in the flask was checked by Giemsa staining every 2 hours. After confirming that the majority of the protozoa had migrated to the ring stage, the culture solution containing the protozoa was diluted with a fresh culture solution (3% hematocrit). The diluted protozoa (0.5 protozoa/0.2 ml) were seeded in 96-well flat bottom plates. Then, half of the culture solution was changed every 48 hours and cultured for 2 weeks. After the culture, the protozoa were detected in about 20 wells per plate. Whether or not each well contained erythrocytes to which a LILRB1 protein binds was screened by flow cytometric analysis. Thereby, F2 clones in which a LILRB1 protein binds and D11 clones in which a LILRB1 protein does not bind were obtained.

(2) Identification of LILRB1 Ligand

The Avi-LILRB1-Fc obtained in Example 1 (1) was biotinylated with BirA biotin ligase. Next, ghost cells of infected erythrocytes were prepared. Specifically, ghost cells of infected erythrocytes were obtained by mixing infected erythrocytes with a low-permeate (40 times the volume of infected erythrocytes) obtained by diluting a RPMI-1640 medium 5-fold with DDW (twice distilled water), incubating the resultant at 4° C. for 15 minutes followed by centrifugation at 15,000 rpm for 15 minutes and washing three times with the low-permeate. It is to be noted that the ghost cells of the infected erythrocytes were prepared using infected erythrocytes infected with protozoa of the F2 and D11 clones in the schizont stage. Then, ghost cells obtained from the infected erythrocytes infected with protozoa of the F2 and D11 clones in the schizont stage were inclubated with biotinized LILRB1-Fc and then cross-linked with 0.25 mmol/l 3,3-dithiobis (sulfosuccinimidyl propionate) (DTSSP, manufactured by Thermo Scientific). The ghost cells were then washed with a phosphate buffer (PBS) and boiled with a sample buffer without 2-mercaptoethanol. After the boil, a coprecipitate containing LILRB1-Fc was obtained from the post-boil sample using streptavidin sephalose. The coprecipitate was eluted at 50 mmol/l DTT (dithiothreitol), trypsinized and subjected to mass spectrometry (LC-MS/MS). MS/MS spectra were analyzed using software (Mascot). As a control, the analysis was performed in the same manner except that ghost cells not treated with biotinylated LILRB1-Fc were used. A similar analysis was performed one more time independently.

As a result, in any of the two independent analyses, an amino acid sequence (FHEYDER (SEQ ID NO: 36)) that matches a partial sequence of a RIFIN protein was obtained only from the infected erythrocytes infected with the F2 clone. These results showed that a RIFIN protein expressed on erythrocytes due to infection with protozoa serves as a ligand of a LILRB1 protein.

(3) Production of Recombinant Protozoa

It is known that multiple types of RIFIN genes exist in Plasmodium falciparum. Therefore, recombinant protozoa expressing various RIFIN genes were produced, and the binding between a LILRB1 protein and a RIFIN protein was examined. Specifically, in order to produce the recombinant protozoa expressing RIFIN genes, the base sequence of corresponding genomic region was specified on the basis of the amino acid sequence of the RIFIN protein. Based on the base sequence of the genomic region, a primer was designed, and the primer was used to PCR-amplify the entire length of each RIFIN gene including introns from the genome of the 3D7 strain. The obtained amplified fragments were inserted into a PfCEN5 expression vector to obtain a PfCEN5 expression vector into which 19 types of RIFIN genes were inserted. Next, the base sequences of the constant region and the variable region of the inserted RIFIN genes were decoded, and it was examined that the base sequences mach 3D7 genome version 3 (release 32, PlasmoDB, http://plasmodb.org). The base sequence of cDNA of the obtained PfCEN5 expression vector was decoded using primers (5′-TTATCCTTATTTTTTAATAACTGCC-3′ (SEQ ID NO: 37) and 5′-GTTCGTGGCATTCCAC-3′ (SEQ ID NO: 38)) specific for the PfCEN5 expression vector. As a result, the introns were accurately spliced in both of the PfCEN5 expression vectors. The RIFIN genes that have been introduced into the PfCEN5 expression vector are as follows.

(RIFIN genes that have been introduced into PfCEN5 expression vector) PF3D7_1254800, PF3D7_0223100, PF3D7_1100400, PF3D7_0632700, PF3D7_0700200, PF3D7_0100200, PF3D7_0900200, PF3D7_0600300, PF3D7_1480000, PF3D7_0100400, PF3D7_0632200, PF3D7_1254400, PF3D7_1479700, PF3D7_0632400, PF3D7_0101000, PF3D7_1000200, PF3D7_0732200, PF3D7_0937500, PF3D7_1300400

In order to produce recombinant protozoa, fresh erythrocytes were transfected with RIFIN-PfCEN5 expression vectors by electroporation, and then the erythrocytes were caused to be infected with a 3D7 strain. Four days after the infection, the infected erythrocytes were cultured in a pyrimesaminecontain-containing RPMI-1640 medium. The recombinant protozoa were then cultured and maintained in a RPMI-1640 medium with 10% human serum and 25 ng/ml pyrimethamine supplemented with human erythrocytes. Control recombinant protozoa were produced in the same manner except that a PfCEN5 expression vector into which a GFP gene had been inserted was used.

(4) Confirmation of Binding Between a LILRB1 Protein and a RIFIN Protein

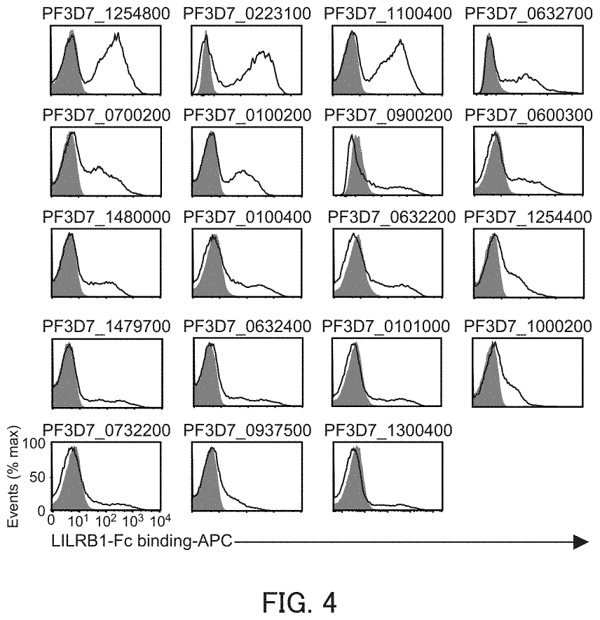

The analysis was performed in the same manner as in Example 2 except that the above-described recombinant protozoa were used in place of the clinical strain. As a control, the analysis was performed in the same manner except that the control recombinant protozoa were used in place of the above-described recombinant protozoa. FIG. 4 shows these results.

FIG. 4 shows histograms illustrating the results of flow cytometry. In FIG. 4, the horizontal axis indicates the binding amount of LILRB1-Fc (LILRB1-Fc binding-APC), and the vertical axis indicates the number of counts. In FIG. 4, histograms indicated by a solid line indicate the results of the present example, and histograms indicated in gray indicate the results of the control. The name on each histogram indicates the name of the RIFIN gene introduced into the recombinant protozoa. As shown in FIG. 4, LILRB1-Fc was bound to all the recombinant protozoa into which the RIFIN genes were introduced. That is, LILRB1-Fc was bound to all of the RIFIN proteins. The LILRB1 was strongly bound in particular to the protein encoded by PF3D7_1254800 (SEQ ID NO: 4), PF3D7_0223100 (SEQ ID NO: 3), PF3D7_1100400 (SEQ ID NO: 6), PF3D7_0632700 (SEQ ID NO: 9), PF3D7_0700200 (SEQ ID NO: 5), and PF3D7_0100200 (SEQ ID NO: 1). Similar results were obtained when a PfCEN5 expression vector having a His-tag added to the C-terminal of the RIFIN gene was used in place of the above-described PfCEN5 expression vector. These results showed that the LILRB1 protein binds to various RIFIN proteins.

### Example 5

The present example examined whether a LILRB1 protein binds to the variable region of a RIFIN protein.

A polynucleotide encoding the conserved region on the N-terminal side (an amino acid region extending from 39th to 139th amino acid residues in the amino acid sequence of SEQ ID NO: 4) or the variable region on the C-terminal side (an amino acid region extending from 166th to 275th amino acid residues in the amino acid sequence of SEQ ID NO: 4) of a RIFIN gene (PF3D7_1254800) and a polynucleotide encoding the transmembrane and intracellular region (an amino acid region extending from 196th to 256th amino acid residues in the amino acid sequence of SEQ ID NO: 39) of PILRα were coupled and the resultant was inserted into a pME18s expression vector. Thereby, an expression vector expressing the fusion protein containing the conserved region of a RIFIN gene and an expression vector expressing the fusion protein containing the variable region of a RIFIN gene were produced. The amino acid sequences of a fusion protein (SEQ ID NO: 40) containing a conserved region and a fusion protein (SEQ ID NO: 41) containing a variable region expressed by the expression vector of the fusion protein are as follows.

Amino acid sequence of PILRa protein
(SEQ ID NO: 39)
MALLISLPGGTPAMAQILLLLSSACLHAGNSERSNRKNGFGVNQPESCS
GVQGGSIDIPFSFYFPWKLAKDPQMSIAWRWKDFHGEFIYNSSLPFIHE
HFKGRLILNWTQGQTSGVLRILNLKESDQTRYFGRVFLQTTEGIQFWQS
IPGTQLNVTNATCTPTTLPSTTAATSAHTQNDITEVKSANIGGLDLQTT
VGLATAAAVFLVGVLGLIVFLWWKRRRQGQKTKAEIPAREPLETSEKHE
SVGHEGQCMDPKENPKDNNIVYASISLSSPTSPGTAPNLPVHGNPQEET
VYSIVKAK
Amino acid sequence of fusion protein containing
conserved region
(SEQ ID NO: 40)
MRAWIFFLLCLAGRALAASDYKDDDDKLECECELYMSNYDNDPEMKRVM
QQFHDRTTQRFHEYDDRMIEKRQKCKDRCNKEIEKIILKDKIEKELTET
FATLNTNITNEDIPTCICKKSVADKIEKTCLKITVGLATAAAVFLVGVL
GLIVFLWWKRRRQGQKTKAEIPAREPLETSEKHESVGHEGQCMDP
Amino acid sequence of fusion protein containing
variable region
(SEQ ID NO: 41)
MRAWIFFLLCLAGRALAASDYKDDDDKLEYETINAFIAKTIEELEGIPG
ITKLFGAKISQFVTPAVFRKPMSLVETILSEKKKLCLCAANKNELLCRG
MNPNVPETLPKKIEVAVNEVLSSVNDTWATATTPTTFFTNPITVGLATA
AAVFLVGVLGLIVFLWWKRRRQGQKTKAEIPAREPLETSEKHESVGHEG
QCMDP

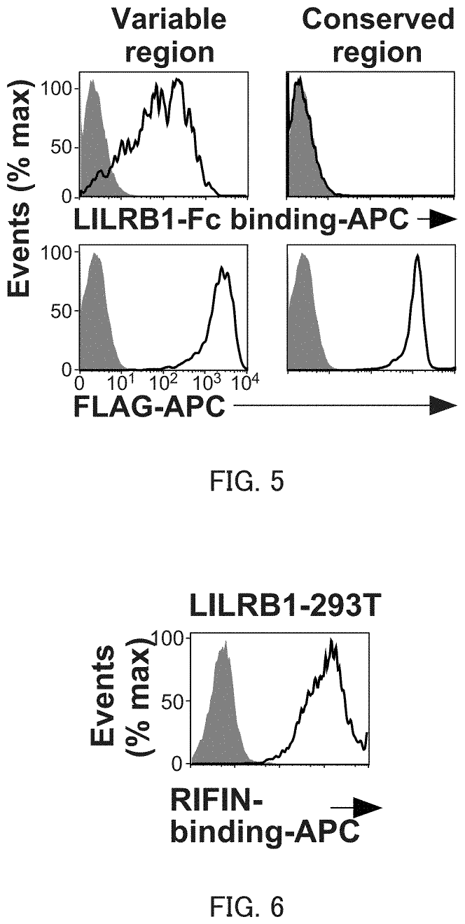

Next, 293T cells were transfected with the expression vector expressing the fusion protein containing the conserved region or the expression vector expressing the fusion protein containing the variable region using the transfection reagent according to the attached protocols. At the same time, the 293T cells were transfected with the expression vector containing the GFP gene. The analysis was performed in the same manner as in Example 2 except that 293T cells after being transceted with the vector were used in place of the infected erythrocytes. As Control 1, the analysis was performed in the same manner except that an APC-labeled anti FLAG antibody was added in place of LILRB1-Fc. As Control 2, the analysis was performed in the same manner except that the vector-free 293T cells were used. FIG. 5 shows these results.

FIG. 5 shows histograms illustrating the results of flow cytometry. In FIG. 5, the horizontal axis indicates the binding amount of LILRB1-Fc (LILRB1-Fc binding-APC) or the binding amount of an APC-labeled anti FLAG antibody (FLAG-APC), and the vertical axis indicates the number of counts. In FIG. 5, histograms show, from the left, the results of the 293T cells transfected with the expression vector expressing a fusion protein containing a variable region (Variable region) and the results of the 293T cells transfected with an expression vector expressing a fusion protein containing a conserved region (Conserved region). In FIG. 5, histograms indicated by a solid line indicate the results of the present example or Control 1, and histograms indicated in gray indicate the results of Control 2. FIG. 5 shows GFP positive cells gated. As shown in the lower histograms of FIG. 5, the results of staining with an APC-labeled anti FLAG antibody showed that the fusion protein containing a conserved region and the fusion protein containing a variable region were expressed to the same extent. As shown in the upper histograms of FIG. 5, LILRB1-Fc was bound to the fusion protein containing a variable region, whereas LILRB1-Fc was hardly bound to the fusion protein containing a conserved region. These results showed that the LILRB1 protein more likely binds to the variable region of RIFIN protein. That is, the LILRB1 protein was found to bind more specifically to the variable region of RIFIN protein compared to the conserved region of RIFIN protein.

### Example 6

The present example examined whether a RIFIN protein binds to a LILRB1 protein.

(1) Preparation of Recombinant RIFIN Protein A recombinant RIFIN protein having a His tagged added to the C-terminal was prepared as follows. First, codon-optimization was performed on a polynucleotide encoding the amino acid sequence of the variable region on the C-terminal side (an amino acid region extending from 166th to 275th amino acid residues in the amino acid sequence of SEQ ID NO: 4) of RIFIN (PF3D7_1254800). The codon-optimized polynucleotide was then inserted into a pET-15b expression vector capable of being added with a His-tag on the N-terminal. Using the obtained pET-15b expression vector, transformation of E. Coli BL21 (DE3) was performed by a conventional method. The obtained transformant was cultured in the presence of IPTG (isopropyl-β-thiogalactopyranoside) to express a recombinant RIFIN protein. After the expression, the transformant was collected and crushed, and a recombinant RIFIN protein was purified from the crush using TALON metal affinity chromatography. The purified recombinant RIFIN protein was then refolded.

(2) Preparation of LILRB1 Protein-Expressing 293T Cell

A polynucleotide encoding LILRB1 was inserted into a pMXs expression vector. The 293T cell was then transfected with an expression vector expressing a LILRB1 protein using the transfection reagent accoridng to the attached protocols. Thereby, a LILRB1 protein-expressing 293T cell was obtained.

(3) Binding Between Recombinant RIFIN Protein and LILRB1 Protein

The LILRB1 protein-expressing 293T cells and the purified recombinant RIFIN protein were mixed and incubated. After the incubation, the resultant was stained with an APC-labeled anti-His antibody (clone 28-75, manufactured by WAKO). The resulting sample was analyzed by flow cytometry. FIG. 6 shows these results.

FIG. 6 shows histograms illustrating the results of flow cytometry. In FIG. 6, the horizontal axis indicates the binding amount of RIFIN (RIFIN-binding-APC), and the vertical axis indicates the number of counts. As shown in FIG. 6, a recombinant RIFIN protein was binding to LILRB1. This result showed that a RIFIN protein binds to a LILRB1 protein.

### Example 7

The present example examined whether a RIFIN protein induces a signal through a LILRB1 protein.

(1) Production of Reporter Cell

A LILRB1 reporter cell was prepared in the same manner as in Reference 2 below. Specifically, the LILRB1 reporter cell was obtained by retroviral gene transfer of a fusion protein obtained by fusing PILRβ with the extracellular domains of NFAT-GFP, FLAG-tagged DAP12, and LILRB1 to a murine T-cell hybridoma.
Reference 2: S hiroishi, M. et al., “Efficient leukocyte Ig-like receptor signaling and crystal structure of disulfide-linked HLA-G dimer.”, J. Biol. Chem., 2006, vol.281, pages 10439-10447

(2) Reporter Assay

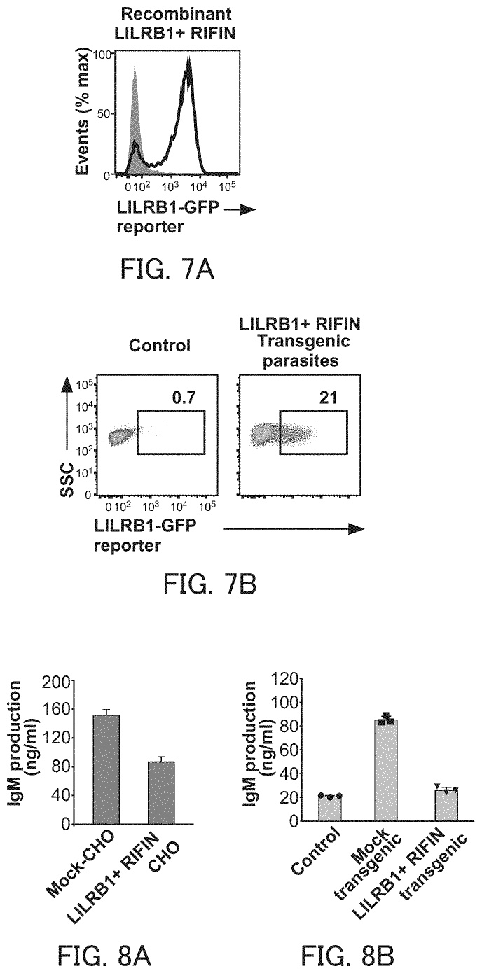

The recombinant RIFIN protein (10 μg/ml) produced in Example 6(1) was immobilized on 96-well plates. Then, the LILRB1 reporter cells were seeded so as to be 1×10⁵ cells/wells and cultured for 16 hours. The culture was carried out at 37° C. under 5% CO₂ atmosphere. After the culture, the expression of GFP caused by a LILRB1 signal was analyzed by flow cytometry. As Control 1, the analysis was performed in the same manner except that a recombinant RIFIN protein was not immobilized. Furthermore, the analysis was performed in the same manner except that recombinant protozoa transfected with the vector encoding PF3D7_1254800 of Example 4(3) were used in place of the recombinant RIFIN protein. As Control 2, the analysis was performed in the same manner except that no recombinant protozoa were added and only reporter cells were used. FIG. 7 shows these results.

FIG. 7 shows graphs illustrating the results of flow cytometry. In FIG. 7, (A) shows the results using the recombinant RIFIN protein, and (B) shows the results using the recombinant protozoa. In (A) of FIG. 7, the horizontal axis indicates the expression level of GFP (LILRB1-GFP reporter), the vertical axis indicates the number of counts, histograms indicated by a solid line indicate results of the present example, and histograms indicated in gray indicate the results of Control 1. In (B) of FIG. 7, the horizontal axis indicates the expression level of GFP (LILRB1-GFP reporter), and the vertical axis indicates the side scattered light (SSC). As shown in (A) of FIG. 7, the expression of GFP was not induced in Control 1, whereas the expression of GFP was induced in the group to which the recombinant RIFIN proteins had been added. In addition, as shown in (B) of FIG. 7, the expression of GFP was not induced in Control 2 (Control), whereas GFP was induced in the group to which the recombinant protozoa had been added. These results showed that a RIFIN protein induces a signal through a LILRB1 protein.

### Example 8

The present example examined whether a RIFIN protein inhibits the activation of B cells through a LILRB1 protein.

(1) Production of RIFIN Protein-Expressing CHO Cell

Using the transfection reagent, CHO cells (puuchased from RIKEN Cell Bank) were transfected with the expression vector expressing the fusion protein containing the variable region of Example 5 and the expression vector expressing CD8 according to the attached protocols. Then, CD8-positive RIFIN-positive cells were isolated using autoMACS® Pro Separator (manufactured by Miltenyi Biotec).

(2) Measurement of B Cell Activity

Peripheral blood mononuclear cells (PBMC) were separated from healthy human blood by Ficoll density gradient centrifugation. Next, PBMC and CD8-positive RIFIN-positive cells were mixed at a ratio of 1:2 (cell number ratio) and cultured for 24 hours. The culture was carried out at 37° C. under 5% CO₂ atmosphere. After the culture, PBMC was stimulated with K3CpG (2 μg/ml) for 3 days. After the stimulation, the culture supernatant was collected, and the IgM concentration in the culture supernatant was measured by ELISA. As Control 1, the measurement was performed in the same manner except that the expression vector containing a fusion protein obtained by fusing human MDA5 (human melanoma differentiation-associated protein 5) in place of the transmembrane region of PILRα. In addition, the measurement was performed in the same manner except that PBMC and the infected erythrocytes infected with the recombinant malaria were mixed at a ratio of 1:100 (cell number ratio), and cultured for 16 hours. As Control 2, the measurement was performed in the same manner except that PBMC alone was used and not stimulated with K3CpG. As Control 3, the measurement was performed in the same manner except that GFP-introduced recombinant malaria was used in place of the aforementioned recombinant malaria. FIG. 8 shows these results.

FIG. 8 shows graphs illustrating the production amount of IgM. In (A) and (B) of FIG. 8, the horizontal axis indicates the type of the sample, and the vertical axis indicates the production amount of IgM. As shown in (A) of FIG. 8, when co-cultured with CHO cells (LILRB1+RIFIN-CHO) expressing a RIFIN protein on the cell membrane, the production amount of IgM decreased compared with Control 1 (Mock-CHO) not expressing a RIFIN protein on the cell membrane. Further, as shown in (B) of FIG. 8, when co-cultured with the recombinant protozoa (LILRB1+RIFIN-recombinant) expressing a RIFIN protein, the production amount of IgM was decreased as compared with Control 3 (Mock-recombinant) not expressing a RIFIN protein, and the production amount of IgM was equivalent to that of the background (Control 2 (Control)). B cells are known to express a LILRB1 protein. Therefore, these results showed that a RIFIN protein inhibits the activation of B cells through a LILRB1 protein.

### Example 9

The present example examined whether a RIFIN protein inhibits the activation of NK cells through a LILRB1 protein.

(1) Production of RIFIN Protein-Expressing K562 Cell

K562 cells expressing a RIFIN protein was established by a retroviral gene expression system with reference to Reference 3 below. Specifically, a polynucleotide encoding the fusion protein was excised from an expression vector expressing the fusion protein containing the variable region of Example 5 and inserted into a pMX-expressing vector. The obtained pMX-expressing vector and PLAT-E retroviral packaging cells were used to prepare a retrovirus containing the expression vector. The K562 cells (purchased from the Institute of Aging Medicine, Tohoku University) were caused to be infected with the retrovirus to produce the K562 cells expressing a RIFIN protein (RIFIN-K562).
Reference 3: Morita, S. et. al., “Plat-E: an efficient and stable system for transient packaging of retroviruses.”, Gene therapy, 2000, vol. 7, pages 1063-1066

(2) Measurement of NK Cell Activity

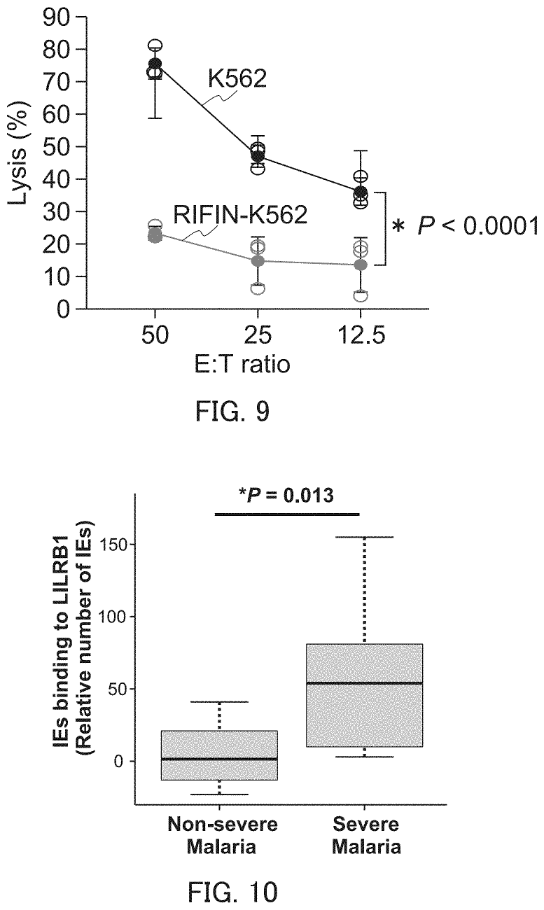

RIFIN-K562 was labeled by culturing at 37° C. for 30 minutes in the presence of a complete medium (composition: phenol-red-free RPMI-1640 medium with 10% heat-inactivated FCS) containing 15 μmol/l Calcein AM (manufactured by Thermo Fisher Scientific). After the labeling, the resultant was washed twice with the complete medium and suspended with the complete medium so that RIFIN-K562 was 5×10³/100 μl. The NK cell line NKL cells (obtained from Dr. Lanier, University of California) were washed twice with a complete medium. After the washing, NKL cells and RIFIN-K562 were seeded in 96-well plate at E:T ratios (NK cell line cell number: RIFIN-K562 cell number) of 50:1, 25:1, or 12.5:1. After the seeding, the plate was centrifuged at 100×g for 5 minutes and further cultured at 37° C. under 5% CO₂ atmosphere for 4 hours. The plate was then centrifuged at 1500 rpm for 2 minutes and the fluorescence of the culture supernatant was measured by TriStar LB941 (manufactured by Berthold Technologies) (experimental release). Furthermore, the maximum release was measured by treating RIFIN-K562 with 2% Triton X-100-containing complete medium. As a control, the measurement was performed in the same manner except that NKL cells were not seeded (spontaneous release). The NK cell activity (dissolution rate) was calculated by the following formula (1). As a control, the calculation was performed in the same manner except that K562 cells were used in place of RIFIN-K562. FIG. 9 shows these results. It was examined by flow cytometry that the NKL cell line expresses LILRB1.

Dissolution rate=(experimental release−spontaneous release)/(maximum release−spontaneous release) (1)

FIG. 9 is a graph illustrating the NK cell activity. In FIG. 9, the horizontal axis indicates the E:T ratio, and the vertical axis indicates the dissolution rate (Lysis (%)). As shown in FIG. 9, when co-cultured with K562 cells expressing a RIFIN protein, the dissolution rate of NK cells was decreased compared with controls. That is, the activation of NK cells was inhibited by a RIFIN protein. These results showed that that a RIFIN protein inhibits the activation of NK cells through a LILRB1 protein.

### Example 10

The present example examined whether the binding of LILRB1 protein is higher in a severe malaria patient than in a mild malaria patient (non-severe malaria patient).

Blood was collected from patients with severe malaria (n=9) or with mild malaria (n=30) in Tanzania. After the blood collection, the blood was seeded on a petri dish in which LILRB1-Fc or LILRA2-Fc (control) was immobilized, and cultured. The petri dish was washed within 24 hours after the start of the culture to remove infected erythrocytes infected with protozoa not binding to the petri dish. Infected erythrocytes infected with protozoa binding to petri dish were immobilized by glutaraldehyde, followed by Giemsa staining and counting. For each patient, the number of infected erythrocytes infected with protozoa binding to the petri dish in which LILRA2-Fc was immobilized was subtracted from the number of infected erythrocytes infected with protozoa binding to the petri dish in which LILRB1-Fc was immobilized to calculate the number of infected erythrocytes binding to LILRB1-Fc. The severe malaria patients included those with cerebral malaria and those with severe anemia. The patients with cerebral malaria were defined as Blantyre coma score<3, and the patients with severe anaemia were defined as blood haemoglobin<5 g/dl. Blantyre coma score and blood haemoglobin were calculated by the methods described above. In addition, mild malaria patients were defined as non-severe malaria patients. FIG. 10 shows these results.

FIG. 10 is a graph illustrating the number of infected erythrocytes binding to LILRB1-Fc. In FIG. 10, the horizontal axis indicates the type of patients, and the vertical axis indicates the number of infected erythrocytes binding to LILRB1-Fc (IEs binding to LILRB1(Relative number of IESs). As shown in FIG. 10, severe malaria patients (severe Malaria) had significantly higher numbers of infected erythrocytes binding to LILRB1-Fc compared to mild malaria patients (non-severe Malaria). These results showed that the binding of LILRB1 protein was higher in severe malaria patients than in mild malaria patients. That is, it was presumed that, since the RIFIN protein that binds to the LILRB1 protein is well expressed in the infected erythrocytes of severe malaria patients, the LILRB1 protein and the infected erythrocytes are interacted, and this involved in severe malaria. This presumption does not limit the present invention by any means.

### Example 11

The present example examined whether inhibition of the interaction between a LILRB1 protein and a RIFIN protein is critical in preventing malaria from becoming severe.

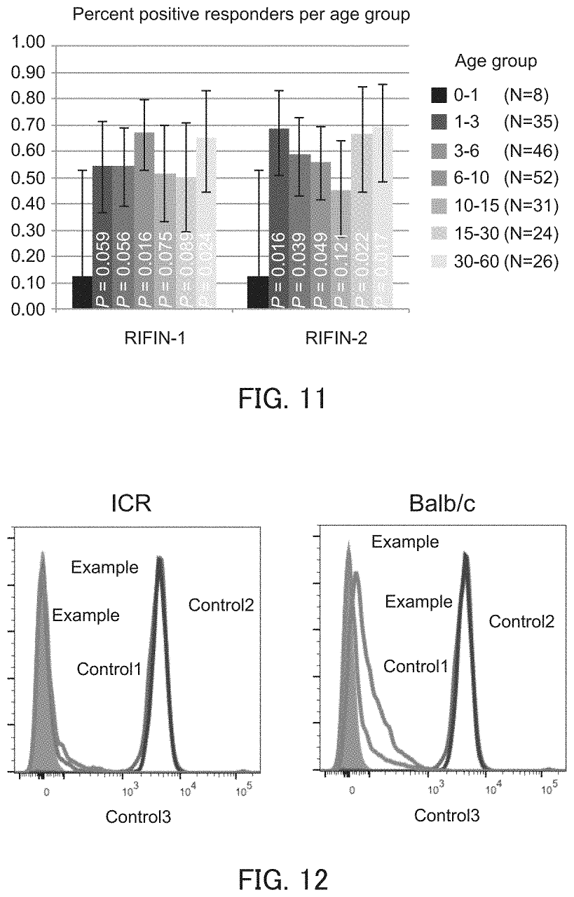

The recombinant RIFIN proteins were immobilized on beads. Next, the immobilized beads were reacted with plasma from 222 Tanzanians, respectively. After the reaction, the binding of IgG to the immobilized beads was analyzed by Luminex (manufactured by Thermo Fisher Scientific Co., Ltd.), and the percentage of Tanzanians with RIFIN protein-binding IgG was calculated (RIFIN-1). The cut-off value was 2SD of the mean value of 43 European donors who have never had malaria. The calculation was performed in the same manner as described above except that the recombinant RIFIN protein of the variable region of PF3D7_1254200 was used in place of the aforementioned recombinant RIFIN protein (RIFIN-2). The percentage was calculated for each age. FIG. 11 shows these results.

FIG. 11 is a graph illustrating the percentage of Tanzanians with RIFIN protein-binding IgG. In FIG. 11, the horizontal axis indicates the type of recombinant RIFIN protein and age, and the vertical axis indicates the percentage of Tanzanians with RIFIN protein-binding IgG. The bars show, from the left, the results in Tanzanians in age up to 1 year (0-1), over 1 year to 3 years (1-3), over 3 years to 6 years (3-6), over 6 to 10 years (6-10), over 10 to 15 years (10-15), over 15 to 30 years (15-30), and over 30 to 60 years (30-60). As shown in FIG. 11, IgG antibodies to the two types of the RIFIN protein were present at equivalent levels in any age ranges. It was also found that IgG antibodies to RIFIN proteins were acquired as early as 1 to 3 years old.

These results suggest that RIFIN proteins are important in the early phase of infection and are important targets in host protective immunity. This led us to presume that the malaria can be prevented from becoming severe by inhibiting the interaction between a LILRB1 protein and a RIFIN protein in living organisms. This presumption does not limit the present invention in any way.

### Example 12

The present example examined whether an anti RIFIN antibody can inhibit the interaction between a LILRB1 protein and a RIFIN protein. The present example also examined whether an anti RIFIN antibody can inhibit a signal through a LILRB1 protein by a RIFIN protein.

(1) Preparation of Anti RIFIN Antibody-Containing Serum

In order to produce an anti RIFIN antibody, the recombinant RIFIN protein was mixed with an adjuvant (manufactured by TiterMax Gold, TiterMax USA, Inc.) and immunized mice threwith (Balb/c (n=2) and ICRs (n=2)). Specifically, each of 6-week-old female Balb/c and ICR mice were immunized with 50 μg of the recombinant RIFIN protein in an animal resource center for infectious diseases. Blood was collected after 2 weeks to obtain a serum.

In order to check the presence or absence of an anti RIFIN antibody in a murine serum, the binding to the beads in which the recombinant RIFIN protein was immobilized (RIFIN beads) was examined. Specifically, RIFIN beads were made by coupling the recombinant RIFIN proteins to Aldehyde/Sulfate Latex beads (3.8 manufactured by invirtogen, Inc., A37304). Then, the RIFIN beads and each serum diluted 100-fold with PBS were incubated for 15 minutes. After washing with PBS, the resultant was stained with an anti mouse IgG antibody (manufactured by Jackson, 5 μg/ml) for 15 minutes. After the staining, RIFIN beads were analyzed by flow cytometry. As a result, it was examined that all sera contained an antibody that binds to a RIFIN protein, i.e., anti RIFIN antibody.

(2) Inhibition of Binding Between RIFIN Protine and LILRB1 Protain by Anti RIFIN Antibody

In order to check whether an anti RIFIN antibodiy inhibits the interaction between a RIFIN and a LILRB1 protein, whether an anti RIFIN antibody-containing serums can inhibit the binding of LILRB1-Fc to RIFIN beads was analyzed. Specifically, the RIFIN beads and each serum diluted 100-fold with PBS were incubated for 15 minutes. After washing with PBS, the resultant was stained with LILRB1-Fc complexed with an APC-labeled anti IgG Fc antibodiy (10 μg/ml) for 15 minutes as described above. After the staining, RIFIN beads were analyzed by flow cytometry. As Control 1, the analysis was performed in the same manner as described above except that the serum was not added. As Control 2, the analysis was performed in the same manner as described above except that a serum from mice not immunized with recombinant RIFIN proteins was used. As Control 3, the analysis was performed in the same manner except that only an APC-labeled anti IgG Fc antibody not forming a complex was used. FIG. 12 shows these results.

FIG. 12 shows histograms illustrating the results of flow cytometry. In FIG. 12, the horizontal axis indicates the binding amount of LILRB1-Fc, and the vertical axis indicates the number of counts. As shown in FIG. 12, the binding between a RIFIN protein and a LILRB1 protein was observed in Controls 1 and 2 not containing an anti RIFIN antibody. In contrast, in the case of adding a serum containing an anti RIFIN antibody, the binding between a RIFIN protein and a LILRB1 protein was inhibited, and the binding was comparable to that of a background (Control 3). These results showed that an anti RIFIN antibody can inhibit the binding between a RIFIN protein and a LILRB1 protein.

(3) Inhibition of Signal Through LILRB1 Protein by RIFIN Protein Due to Anti RIFIN Antibody

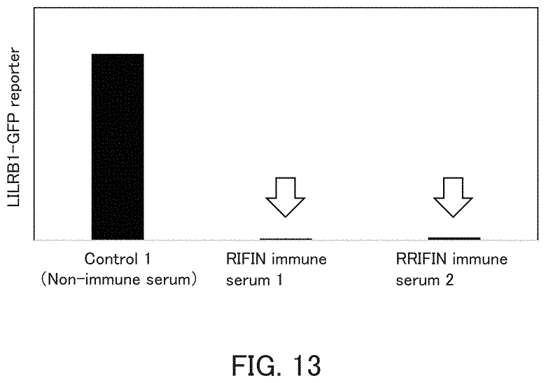

The recombinant RIFIN protein (10 μg/ml) was immobilized on 96-well plates. Then, the LILRB1 reporter cells were seeded so as to be 1×10⁵ cells/wells. In addition, the serum was added to each well at 100-fold dilution and cultured for 18 hours. The culture was performed at 37° C. under 5% CO₂ atmosphere. After the culture, the expression of GFP caused by a LILRB1 signal was analyzed by flow cytometry. As a control, the analysis was performed in the same manner as described above except that a serum from mice not immunized with recombinant RIFIN proteins was used. FIG. 13 shows these results.

FIG. 13 is a graph illustrating the results of flow cytometry. In FIG. 13, the horizontal axis indicates the type of sample, and the vertical axis indicates the expression level of GFP (LILRB1-GFP reporter). As shown in FIG. 13, the expression of GFP was induced in a control, whereas the expression of GFP was hardly induced when the serum containing an anti RIFIN antibody was added. In addition, as described above, an anti RIFIN antibody inhibits the binding between a RIFIN protein and a LILRB1 protein. Therefore, these results showed that an anti RIFIN antibody can inhibit the binding between a RIFIN protein and a LILRB1 protein, thereby inhibiting a signal through a LILRB1 protein by a RIFIN protein.

As described above, it is presumed that a RIFIN protein induces the malaria becoming severe by binding to a LILRB1 protein in the living organism. Moreover, it is considered that the severe malaria is due to the fact that a RIFIN protein inhibits the activation of immune cells such as B cells and NK cells by a signal through a LILRB1 protein. As described above, a binding inhibitor that inhibits the binding between a LILRB1 protein and a RIFIN protein such as an anti RIFIN antibody can inhibit a signal through a LILRB1 protein by a RIFIN protein. Therefore, by directly or indirectly inhibiting the binding between a RIFIN protein and a LILRB1 protein by the binding inhibitor, the inducer, and the expression inhibitor according to the present invention, it is possible to inhibit malaria becoming severe in a living organism.

While the present invention has been described above with reference to illustrative embodiments and examples, the present invention is by no means limited thereto. Various changes and variations that may become apparent to those skilled in the art may be made in the configuration and specifics of the present invention without departing from the scope of the present invention.

This application claims priority from Japanese Patent Application No. 2017-228226 filed on Nov. 28, 2017. The entire subject matter of the Japanese Patent Applications is incorporated herein by reference.

(Supplementary Notes)

Some or all of the above embodiments and examples may be described as in the following Supplementary Notes, but are not limited thereto.

### (Supplementary Note 1)

An antimalarial drug including:
a binding inhibitor that inhibits binding between a RIFIN protein and a leukocyte immunoglobulin-like receptor subfamily B member 1 (LILRB1) protein;
an inducer of the binding inhibitor; or
an expression inhibitor of RIFIN or LILRB1.

### (Supplementary Note 2)

The antimalarial drug according to Supplementary Note 1, wherein
the binding inhibitor is at least one of a binding substance that binds to the RIFIN protein or a binding substance that binds to the LILRB1 protein.

### (Supplementary Note 3)

The antimalarial drug according to Supplementary Note 2, wherein
the binding substance that binds to the RIFIN protein binds to a variable region of the RIFIN protein.

### (Supplementary Note 4)

The antimalarial drug according to Supplementary Note 2 or 3, wherein
the binding substance includes an antibody or an antigen-binding fragment thereof.

### (Supplementary Note 5)

The antimalarial drug according to any one of Supplementary Notes 1 to 4, wherein
the inducer includes the RIFIN protein or a part of the RIFIN protein, or a nucleic acid encoding them.

### (Supplementary Note 6)

The antimalarial drug according to Supplementary Note 5, wherein
the part of the RIFIN protein includes the variable region of the RIFIN protein.

### (Supplementary Note 7)

The antimalarial drug according to any one of Supplementary Notes 1 to 6, wherein
the RIFIN protein includes a LILRB1-binding RIFIN protein.

### (Supplementary Note 8)

The antimalarial drug according to any one of Supplementary Notes 1 to 7, including: the inducer of the binding inhibitor and an adjuvant.

### (Supplementary Note 9)

The antimalarial drug according to Supplementary Note 1, wherein
the expression inhibitor is at least one selected from the group consisting of a substance that inhibits an expression of mRNA of a RIFIN gene or a LILRB1 gene, a substance that cleaves expressed mRNA, and a substance that inhibits translation of a protein from the expressed mRNA.

### (Supplementary Note 10)

A malaria treatment method, including:
administering the antimalarial drug according to any one of Supplementary Notes 1 to 9 to a patient.

### (Supplementary Note 11)

A method for screening a candidate substance for malaria treatment, including:
selecting, as a candidate substance for malaria treatment, a binding inhibitor that inhibits binding between a RIFIN protein and a leukocyte immunoglobulin-like receptor subfamily B member 1 (LILRB1) protein, an inducer of the binding inhibitor; or an expression inhibitor of RIFIN or LILRB1 from a test substance.

### (Supplementary Note 12)

The screening method according to Supplementary Note 11, including:
detecting binding between the RIFIN protein and the LILRB1 protein in a presence of the RIFIN protein, the LILRB1 protein, and the test substance; and
selecting, as the candidate substance for malaria treatment, the test substance that inhibits binding between the RIFIN protein and the LILRB1 protein.

### (Supplementary Note 13)

The screening method according to Supplementary Note 11, including:
administering the test substance to a living organism;
collecting a biological sample from the living organism;
detecting binding between the RIFIN protein and the LILRB1 protein in the presence of the RIFIN protein, theLILRB1 protein, and the biological sample; and
selecting, as the candidate substance for malaria treatment, the test substance that inhibits binding between the RIFIN protein and the LILRB1 protein.

### (Supplementary Note 14)

The screening method according to Supplementary Note 11, including:
bringing the test substance into contact with the RIFIN protein or the LILRB1 protein;
detecting binding between the RIFIN protein or the LILRB1 protein and the test substance; and
selecting, as the candidate substance for malaria treatment, the test substance binding to the RIFIN protein or the LILRB1 protein.

### (Supplementary Note 15)

The screening method according to Supplementary Note 11, including:
causing the test substance to be coexist in an expression system of the RIFIN or the LILRB1 to express the RIFIN or the LILRB1;
detecting an expression of the RIFIN or the LILRB1 in the expression system; and
selecting, as the candidate substance for malaria treatment, the test substance with which the expression level of the RIFIN or the LILRB1 is lower than that of a control expression system in which the test substance is not present.

### (Supplementary Note 16)

The screening method according to any one of Supplementary Notes 11 to 15, wherein
the test substance is at least one selected from the group consisting of a low molecular weight compound, a peptide, a protein, and a nucleic acid.

### (Supplementary Note 17)

A malaria severity marker, wherein the marker is RIFIN.

### (Supplementary Note 18)

The malaria severity marker according to Supplementary Note 17, wherein
the RIFIN is a leukocyte immunoglobulin-like receptor subfamily B member 1 (LILRB1)-binding RIFIN.

### (Supplementary Note 19)

A method for testing a risk of severe malaria, including:
measuring an expression of RIFIN in a biological sample of a subject.

### (Supplementary Note 20)

The test method according to Supplementary Note 19, wherein
an expression level of RIFIN is measured in the measuring.

### (Supplementary Note 21)

The test method according to Supplementary Note 20, including
testing the subject for a risk of severe malaria by comparing the expression level of RIFIN in the biological sample of the subject with a reference value, wherein
the reference value is an expression level of RIFIN in a biological sample of a healthy subject or an expression level of RIFIN in a biological sample of a severe malaria patient.

### (Supplementary Note 22)

The test method according to Supplementary Note 21, wherein in testing, it is determined the subject is at a risk of severe malaria when the expression level of RIFIN in the biological sample of the subject is higher than the expression level of RIFIN in the biological sample of the healthy subject, when the expression level of RIFIN in the biological sample of the subject is the same as the expression level of RIFIN in the biological sample of the severe malaria patient, or when the expression level of RIFIN in the biological sample of the subject is higher than the expression level of RIFIN in the biological sample of the severe malaria patient.

### (Supplementary Note 23)

The test method according to any one of Supplementary Notes 19 to 22, wherein
the biological sample includes blood.

### (Supplementary Note 24)

The test method according to any one of Supplementary Notes 19 to 23, wherein
the biological sample includes erythrocytes.

### (Supplementary Note 25)

The test method according to any one of Supplementary Notes 19 to 24, wherein
the RIFIN is a leukocyte immunoglobulin-like receptor subfamily B member 1 (LILRB1)-binding RIFIN.

### (Supplementary Note 26)

The test method according to any one of Supplementary Notes 19 to 25, wherein
the expression of RIFIN is at least one of an expression of a RIFIN protein or an expression of a RIFIN mRNA.

### (Supplementary Note 27)

A test reagent for use in the test method according to any one of Supplementary Notes 19 to 26, including:
a reagent for measuring an expression of RIFIN.

### (Supplementary Note 28)

The test reagent according to Supplementary Note 27, wherein
the expression measuring reagent includes a binding substance that binds to a RIFIN protein and a detecting reagent that detects binding between the RIFIN and the binding substance.

### (Supplementary Note 29)

The test reagent according to Supplementary Note 27, wherein
the expression measuring reagent is a reagent for amplifying a RIFIN gene mRNA by reverse transcription.

### INDUSTRIAL APPLICABILITY

As described above, according to the antimalarial drug of the present invention, malaria can be treated. Thus, the present invention is extremely useful, for example, in the clinical field.

1-8. (canceled)

9. A method, comprising:
administering an antimalarial drug to a malaria patient to prevent malaria from becoming severe or treat malaria, wherein
the antimalarial drug is a binding inhibitor that inhibits binding between a RIFIN protein and a leukocyte immunoglobulin-like receptor subfamily B member 1 (LILRB1) protein;
an inducer of the binding inhibitor; or
an expression inhibitor of RIFIN or LILRB 1.

10. A method for screening a candidate substance for malaria treatment, comprising:
selecting, as a candidate substance for malaria treatment, a binding inhibitor that inhibits binding between a RIFIN protein and a leukocyte immunoglobulin-like receptor subfamily B member 1 (LILRB1) protein, an inducer of the binding inhibitor; or an expression inhibitor of RIFIN or LILRB 1 from a test substance.

11-12. (canceled)

13. A method, comprising:
measuring an expression of RIFIN in a biological sample of a subject to test a risk of severe malaria.

14. The method according to claim 13, wherein
the RIFIN is a leukocyte immunoglobulin-like receptor subfamily B member 1 (LILRB 1)-binding RIFIN.

15. (canceled)

16. The method according to claim 9, wherein
the binding inhibitor is at least one of a binding substance that binds to the RIFIN protein or a binding substance that binds to the LILRB1 protein.

17. The method according to claim 16, wherein
the binding substance that binds to the RIFIN protein binds to a variable region of the RIFIN protein.

18. The method according to claim 16, wherein
the binding substance comprises an antibody or an antigen-binding fragment thereof.

19. The method according to claim 9, wherein
the inducer comprises the RIFIN protein or a part of the RIFIN protein, or a nucleic acid encoding them.

20. The method according to claim 19, wherein
the part of the RIFIN protein comprises the variable region of the RIFIN protein.

21. The method according to claim 9, wherein
the RIFIN protein comprises a LILRB1-binding RIFIN protein.

22. The method according to claim 9, wherein
the antimalarial drug comprises the inducer of the binding inhibitor and an adjuvant.

23. The screening method according to claim 10, comprising:
detecting binding between the RIFIN protein and the LILRB1 protein in a presence of the RIFIN protein, the LILRB1 protein, and the test substance; and
selecting, as the candidate substance for malaria treatment, the test substance that inhibits binding between the RIFIN protein and the LILRB1 protein.

24. The method according to claim 13, wherein
the biological sample comprises erythrocytes.

25. The method according to claim 14, wherein
measuring the expression of LILRB1-binding RIFIN in the biological sample using a LILRB1 protein.


SEQUENCE LISTING (41 sequences)

SEQ ID NO: 1 - protein, 331 aa, Plasmodium falciparum
MKIHYINILLFELPLNILIYNQRNHKSTTPHTPNHTQTTRLLCECELYSPANNDNDAEMKRVMQQFEDRTTQRFHEYDERMKTTRQKCKEQCDKEIQKIILKDKLEKELMDKFATLQTDIQSDSIPTCICEKSLEDKVEKGCLRCAGVLGGGIAPGWSLVSGLGYAVWTNYVTQTALQKGIEAGVKAGIEGLRDFSGLGKLIPISVIQNLINHTNYDIAKTYITFVKSVNSTKCAVKEHSFCFSTYISNENALSKRAAGIAEYAADMAKITERGVLDAATPGLTTYSNAITASVVAIVVIVLVMIIIYLILRYRRKKKMKKKLQYIKLLEE

SEQ ID NO: 2 - protein, 376 aa, Plasmodium falciparum
MKVHYINILLFALPLNILIYNQRNHNSTTHHTLKIPITRLLCECELYELANYDNDPEMKEVMQQFEDRTTQRFHEYDERMKTTRQKCKDKCDKEIQKIILKDKLEKELMDKFATLHTDIQSDAIPTCICEKSLEDKMEKECLKCAQNLGGIVAPSTGVLGEIAALAVNAWKTEAIAAATKAAIAKGTAKGLAAGAAKGVAEVIAQVESQFRLSTIGVKELGSIFNASNYTNETFISGYIYAQYQGSQCGSLSMLLGKSKPFCTFVEGRIFATSVRVGRSFSPEDFIKTTVQTIVKNAKTTAEATKAQVASAEKAAVLETSKKAIEATTTPYYTPIIVSIVAIVVIILIMVIIYKILRYRRKKKMKKKLQYIKLLKE

SEQ ID NO: 3 - protein, 330 aa, Plasmodium falciparum
MKDHYINILLFALPLNILVYNQRNYYITRTPKATTRTLCECELYAPATYDDDPQMKEVMDNFNRQTQQRFHEYDERMKTTRQKCKDQFDKEIQKIILKDKLEKELMDKFATLQTDIQNDAIPTCICEKSLADKVEKTCLRCGSVFGGGITPGWGLISGLGYVGWTNYITEIAIQKGIEAGVKAGIQELKGFAGLSRLINFSEIKNLINHTNYFKEMTYVSFLQDANKTHCSARPTSKEIFCNFVSHNGESALSKRAAGIADYAADMAKITEEGVLEEGASATSSLTTAIIASIIAIVVIILIMIIIYLVLRYLRKKKMKKKLEYIKLLKE

SEQ ID NO: 4 - protein, 317 aa, Plasmodium falciparum
MKIHYTNILLFPLKLNILVNTHKKPSITPRHIQTTRLLCECELYMSNYDNDPEMKRVMQQFHDRTTQRFHEYDDRMIEKRQKCKDRCNKEIEKIILKDKIEKELTETFATLNTNITNEDIPTCICKKSVADKIEKTCLKYGGALGGGVMPGLGLIGGNSVYILANYETINAFIAKTIEELEGIPGITKLFGAKISQFVTPAVFRKPMSLVETILSEKKKLCLCAANKNELLCRGMNPNVPETLPKKIEVAVNEVLSSVNDTWATATTPTTFFTNPIILSAIAILVIVIIMVIIYLILRYRRKQKIKKKLQYIKLLKE

SEQ ID NO: 5 - protein, 323 aa, Plasmodium falciparum
MKIHYINILLFALPLNILVHNQRNHKKTILHTPKTKSTRTHRSLCECELYAPVNYYSDPQMKEVMDNFNKQTQQRFHEYDERMKTTRQKCKDRCDKDIQKIILKDKIEKELAETFSSLHTDIQSDAIPTCICEKSLADKVEKGCLRCAQNLGGLVPGMGLIGGTAVYAAAVKAATKAGMKEALEGLKSIGGLKLLLQDKFTELVTTRNFQCPNALVGAVQNVINTQCVGPAAKNQLLCNGYEAQNDSRIIQKAVDAGRDGADVYIRTFSDSTTITTFLTDPIVISAIVVISIVVILLIIYLILRYRRKIKMNKKLQYIKLLKE

SEQ ID NO: 6 - protein, 323 aa, Plasmodium falciparum
MKIHYINILLFALPLNILVNNQRNHNNSTYHTSNTKTIKSHRSLCECELYAQSNYENDQEMKDVIKEFNDRTAQRFEEYNERMQVKKDQCKEQCDKEIQQIILKDKIEKELTERFSALETKIDTNDILTCICEKSVTDKFEKTCLKCSGIFATAVPELGLIGGTVVYAAAVKAATKAGMEAALVGLESVNGLRGLLGEKIKDLVTTTNFQCPNALMGLVQNVKDTQCVGAAAQSQVFCKGLLPESTSRIIQKAAAAGREGAEAYNTTFSDSTTITAFLTDPIVISAIVVISIVVILLIIYLILRYRRKIKMNKKLQYIKLLKE

SEQ ID NO: 7 - protein, 340 aa, Plasmodium falciparum
MKVHYINILLFSLPLNILEHNPWNHYMKPHTYTNRSLCECELYELANYDNDPQMKEVMENFIKQTQQRFHEYDERLQSKRKQYKDKCDKEIQKIILKDKLEKQMAQQLTTLDPNITTEDIPTCVCEKSLADKTEKFCLNCGKTMGGVAPGWGLVSGLGYAGWSHYAATTLVKIATDAGIAEGLKVGLTKVTEIVTQLSSSTEVAIPTIDVLTNLTTGISADNVTLLGIFKTINIGMKGEFDTDTYALFSTWVQNIATTPKSYMGRYLTEAEEVTKAFADAQTRVLTQAGNVTSNLTTGITVSIIAIVVIVLVMLIIYLILRYRRKKKMKKKLQYIKLLKE

SEQ ID NO: 8 - protein, 371 aa, Plasmodium falciparum
MKIHYINILLFELPLNILIYNQRNHNSTTPHHPPNTRLLCECELYAPATYDDDPQMKEVMQQFEDRTSQRFHEYDERMKTTRQKCKDKCDKEIQKIILKDKLEKELMDKFATLQTDIQNDAIPTCVCEKSLEDKMEKGCLRCGGVLGGGIAPTFGLIGSVAINMWKTTEIAAATKAAIAAGKAAGKIAGEAAGKKAVIEALKYFGVDDFFPEIFKSILKMSRYTDVTKFGAAIAEKHVLNCAMSARGGSVNDSTCNAFEIKLGLFEAETGKPNGPPAYQAIPQKINELAEEATQAAAEAAKKASESATAAFETAEKEAIEAASMQLYTTIAYSILAILIIVLIMVIIYLILRYRRKKKMKKKLQYIKLLEE

SEQ ID NO: 9 - protein, 376 aa, Plasmodium falciparum
MKIHYINILLFELPLNILIYNQRNHYITRTPKATTRSLSECELYAPSNYDNDPQMKEVMDNFNRQTQQRFHEYDHRMKTTRQKCKEQCDKEIQKIILKDKLEKELMDKFATLHTDIQNDAIPTCVCEKSVADKVEKNCMKCTQNLGGIVAPSSGVLAGIAEGALYVWRDAEIVAAIAAAKEAGAAKGAAVGIKEGIKVLLNRLNTDFGLSPVRIKELESVINGTNYTDVTFIYEAIYTTYKRSCVPVDVSVRFTVADTDLTFCESVWNQTLAVSQRNMGTSPLPIIQKTAQKIVSDANFTAAATAETATEEATTTLTAKNTGEVNATYMGYQTPIIASIVAILVIVLVMIIIYLILRYRRKKKMKKKLQYIKLLEE

SEQ ID NO: 10 - protein, 377 aa, Plasmodium falciparum
MKIHYTNILLFPLKLNILVNTHQKPHTTARHTQKIPTTRSLSECELYAPVNYYSDPQMKEVMDNFNKQTQQRFHEYDERVQNTRQKCKEQCDKEIQKIILKDKIEKELNEKFSALHTDIQSDDIPTCICEMSMADKVEKGCLRCVGVFGGGIAPSVGLLGGLGIYVWKPGALKVAITAALNANSVKIAAAANAAGEAMGVKTVIEGLKALNVHGLCPDLFESIGTKIHYTNAEEIAKIIVAKYRATCNLSTGTSSTQAMCKQFDYTFGMRIRLGSPVEYGPPPASAIPDTVKKVVAGAEQAAEAKAANVRTTISSKIITEETDVINTIYMSNQTAIIASIIAIVIIVLIMVIIYLILRYRRKKKMKKKLQYIKLLEE

SEQ ID NO: 11 - protein, 372 aa, Plasmodium falciparum
MKIHYTNILLFPLKLNILVNTHKKPSITSRHIQTTRLLCECELYTPNYDNDPEMKSVMQQFHDRTTQRFHEYDENLKEKRQKCKDKCDKEIQKIILKDKIEKELTEKFSSLQTDIHSDAIPTCICEKSLADKVEKNCLKCTQNLGKIVAPSSGVLAGISEAALSVWKTTEIAAAMELAKQAGAAAGLKAGHLAGTNAVIEQLRTLGIYFVGDKLLETIIDVTNYMNVSFIYDKVYSHYTTSCTPSLVNDQLVGTFNTSDPFCNLVHSNLQGSFYRSSAQTIIYEKVEEAVAGAEQAATTKTAVMTPIYTTEFTAKNIAEVEAATTSYYTPIIASIVAIVIIVLIMVIIYLILRYRRKMKLKKKLQYIKLLEE

SEQ ID NO: 12 - protein, 379 aa, Plasmodium falciparum
MMLNYTNILLFYLSLNILSSSSEVYNQRNHFITYTPKRSTRLLCECELYTSIFDNDPEMKSLIEHFNKQTQQRFHEYDERMKTTRQKCREQCDKEIQKIILKDKLEKELAEKFVTLQTDIQSDAIPTCICEKSLADKVEKTCLKCGGVLGGGVTPAWGFLSGIVYTGWKAAALAAATKEAIAEGAAKGAAAGTKAGIKAVMDVLYSDFGLSIEGVQKMGLVLSATNYKDVPMITKALYSKFQVSSCLRGGPVPGVPPVRPTDGTFCSAMLEKILAQENVVKQNSLEGSIKSVVNQIVTEAKSAAVSETAKVTASETETLKATNIAAVNATYASSQTAIIASIIAIVVIILIMVIIYLILRYRRKKKMKKKLQYIKLLEE

SEQ ID NO: 13 - protein, 339 aa, Plasmodium falciparum
MKIHYINILLFELPLNILIYNQRNHNSTTHHTLKIPITRLLCECELYAPSNYDNDPEMKEVMEIFDRQTSERFHEYDERMKTTRQKCREQCDKEIEKIILKDKLEKELMDKFATLHTDMQSDAIPTCVCEKSVADKTEKVCLNCGKTMGAVAPAWGLISGLWYATWSQYVSAKILEVGISEGIKEGLTQIMKFTISLYPKANLPNITVTQMLSSGKFTNNVTLFDMVQHINNTMYTTLEAEEYSKFCGVVSSMAKYKNITFNRTYGKYSTAVTEAVTQGKTNAINTLTPATNTLTTAIIASMVAIVVIVLVMIIIYLILRYRRKKKMKKKLQYIKLLEE

SEQ ID NO: 14 - protein, 350 aa, Plasmodium falciparum
MKVHYINILLFVIPLNILINDQRNHKSTTHHTLKIPITRLLCECELYTPANYDNDPQMKEVMDNFNRQTQQRFHEYDERMVEKRMQCKDKCDKEIQKIILKDKLEKELMDKFATLHTDIQSDAIPTCVCEKSVADKMEKGCLRCGSILGAAMPEMGSIGGSLLSALSAWKPVAIEAAEKAAIAKATDLATQAGMREVVLKIEQFLKNFTEKEGLVNFTSVVNKSNFKCPTALFQNANELLSDSCIPDEVTNRTSTFCSTIAYGEKTTFEPFAQAGATTFQETLTAKTPVLQARYTAAVKTAYGGYQTAIIASIVAIVVIVLIMVIIYKILRYRRKKKMKKKLQYIKLLEE

SEQ ID NO: 15 - protein, 372 aa, Plasmodium falciparum
MKIHYTNILLFALPLNILVNTHKKPHTTARHTQKIPTTRSLSECELYAPVNYYSDPQMKEVMDNFNKQTQQRFHEYDERMKTTRQKCKDKCDKEIQKIILKDKLEKELMDKFATLDTDIQSDAIPSCVCEKSIAEKAEKGCLRCGYGLGSVAPMIGLTGSVAVNVWKTAELAAAMELAKQAGAAAGIKAGHLAGTKVVIDQLHTLGIYFVGGKPLESIIHVTNYMNVSVIYDKVYSHYTTLCTPRFVIDRPVGDFIFSGPVCNLVQPNHQGIWVKSSAQAIIKKKVEEAVAEGTQAADVVAKNTADEVTKAAIKTSTEAIDAATTTYYTPIIASIVAIVLIVLIMVIIYKILRYRRKRKMKKKLQYIKLLEE

SEQ ID NO: 16 - protein, 351 aa, Plasmodium falciparum
MKVHYINILLFALPLNILIYNQRNHKSTTHHTLKIPITRLLCECELYTPANYDNDPQMKEVMDNFNRQTQQRFHEYDERMVEKRMQCKDKCDKEIQKIILKDKLEKQMEQQLTTLETKIDTNDIPTCVCEKSMTDKVEKGCLRCGRNLGVAVPGLGVLGAYGAHSIVKVAMATAEKVGIQLGIDAGNAAGIKAVIEALNSSLNIDNLGGITLDTVLKGNNFKNIDFLVYILTDKYNTTCTVSNTEVETLLCYIGKEKPTLPYTLIQSNVRKAVAEATEVATSTTEEMTTIYTTQELSKVTSTGAILSNPIIISFIVIVIVVIIFLIIYLILRYRRKKKTKKKLQYIKLLKE

SEQ ID NO: 17 - protein, 380 aa, Plasmodium falciparum
MKIHYINILLFELPLNILIYNQRNHKSTTHHTLKIPTTRLLCECELYSPANYDNDPEMKEVMEIFDRQTSERFHEYDERMVEKRMQCKDKCDKEIQKIILKDKLEKELAEKFVTLQTDIQNDAIPTCVCEKSIADKVEKGCLRCVGVFGGGVMPGFGTIGGTALYALNQLKPAVFKAAIKAALEEGAAEILAAGIEAGDAAGMNVVRYGLRYLHVHELFPVIFDSFVKTRPYNEITSIANSILLKYGPTCTGLDNNSPPAACTKFQLNLGIHKKIGAMIDTHGTPASTAIRQGLEGILEEATQTAEAAAKIAEKGVAAEITARETALIEAGFNSSITSINASIFAIVVIVLIMVIIYLILRYRRKKKMKKKLQYIKLLEE

SEQ ID NO: 18 - protein, 372 aa, Plasmodium falciparum
MKFSYFNILLFSIPLNILINDQRNHKSTTHHTLKIPITRLLCECELYSPDNYDNDAEMKRVMQQFEDRTSQRFHEYDERMQSKRMQCKDRCDKEIQKIILKDKIEKELSQHLSTLETNIDTNDIPTCVCEKSLADKVEKGCLRCGYGLGTVAPTVGLIGAVAVNELKKAAMAIAIKDAIAEGLVAGETARIQASIKAVILGIKSKFRIDTLGGEVLESIITAQKYDDVSLISESIYMQYQSTCLPQYVGHGADLSKPICHTVYTLDFVQGKVHVPGSLQGSIKKALEKIVAEAKSNAVSETANVTTRQTAVFESRNIAAVDATYASYQTAIVASVVAILVIVLVMLIIYLILRYRRKKKMKKKLQYIKLLKE

SEQ ID NO: 19 - protein, 374 aa, Plasmodium falciparum
MKFSYFNILLFSIPLNILINDHSKYSSCKHTSNSKTTKPHRSLYECGLYSPANNDNDPEMKRVMQQFEDRTSQRFHEYDERMQSKRMQCKEQCDKEIQKIILKDKLEKQMEQQLNTLETKIDTDDIPTCVCEKSLADKVEKGCLRCGYGLGTVAPTVGLIGAVAVHVWKPMALEAAIEAAIAKSAAEISAAANAAGIQAGKIAVIESLKKLYVDYFWPEMSNYILNMSHYNGVANLTAFIHEPKFNVCKDAGEVILDKCNAFDMGFGILKKDGVTNGLLPKDAVPRVLKGIVGQAEGPAKVAADAARQTVTAEITEKETAAINTIFMSKQTAIIASVVAIVVIVLIMIIIYLILRYRRKKKMKKKLQYIKLLKE

SEQ ID NO: 20 - protein, 350 aa, Plasmodium falciparum
MKIHYINILLFPLKLNILIYNQRNHKSTTHHTLKIPITRLLCECELYTPANYDNDPQMKEVMDNFNRQTQQRFHEYDERMVEKRMQCKDKCDKEIQKIILKDKLEKQMEQQLTTLETKITTDDIPTCLCEKSVADKMEKTCLRCAGVLGGGVMPGMGLIDGSLLGAISVLKPAAIIAAKDAALAEATALATQAGMREVVLKIEQFLKLFSEKEKIFDLKLIVNKSNFSCGSSLFQNAKELANKSCVAKPNGSYTSFCNSITYSRVEPFNGYAQAGITKYNETLPLQKALLEKAKVDAVNTTYAAYHTSIIASIVAVVVIVLIMVIIYLILRYRRKKKMKKKLQYIKLLEE

SEQ ID NO: 21 - protein, 343 aa, Plasmodium falciparum
MKIHYTNILLFPLKLNILVNTHKKPHTTARHTQKIPTTRSLSECELYAPVNYYSDPQMKEVMDNFNKQTQQRFHEYDERMKTTRQKCKDKCDKEIQKIILKDKLEKQMAQQFSTLHTDIQSDDIPTCICEKSLADKVEKGCLRCAQNLGGVAPGWGLLSGFGYVTWSQYISGIAAKAAADAGLKAGVKVGLVNAVKIVTKTLDGFGEVPTMDWAKLIAFGDFSDGVTLHAIFKNLNNMMNCYLDSGKYSQFSTVVQKFAENPRSYATPYSTEVTEVTKAVADAKTGVLTKAGNATSSLSTGITASIIAIVVIVLIMVIIYLVLRYRRKKKMKKKLQYIKLLEE

SEQ ID NO: 22 - protein, 366 aa, Plasmodium falciparum
MKDHYINILLFALPLNILVYNQRNYYITPRHTETNRSLCECELYSPTNYDSDPEMKRVMQQFVDRTTQRFHEYDERMKTTRQKCKDKCHKEIEKIILKDKMEKQMAQQLTTLETKIGTDDIPTCVCEKSMADKMEKDCLRCTYGLGTLAPTVGLIGSVAVGAWKPTALKAAIVAAQKAGDAAGVAAGEAAGKKAVILALQHFKLDNLFPEIYNAIVKIRHYADVKNFSVAIVEEHSLKCQSLDLKVTTNPTCETFEFNIGMRIPDSSFVEPVDQVVPEVLDSLVGNIKEVAEAKAAEVAAAKTAEFKIANVGAVESTYGSCQTAIIASIVAIVVIVLIMVIIYLILRYRRKKKMKKKLQYIKLLKE

SEQ ID NO: 23 - protein, 379 aa, Plasmodium falciparum
MKVHYINILLFALPLNILIYNQRNHKSTTHHTLKIPITRLLCECDIYTSIYDNDPQMKEVMDNFNRQTQQRFHEYDERMQGKRQKCKDKCDKEIQKIILKDKLEKELMDKFATLHTDMQSDSIPTCVCEKSVADKVEKNYMKCTQNLGGIVAPSSGVLAGIAELGLSAWKTTALKTAIAAAEQAGAAKGLAAGAAKGATRLIELIQSTFKIQNIAGKSLGTFIDATNYNNGPFIYQAIYTKFEMSLCLPVFPGVDPVPGAVRDPTFCNLFEKFVPTNGSSNRDSIINAIETYVQPFVSDAKFTAAATAETATEEATAVLITKKTGEVTTTYASYQTAIIASIVAILVIVLVMIIIYLILRYRRKKKMKKKLQYIKLLEE

SEQ ID NO: 24 - protein, 349 aa, Plasmodium falciparum
MKIHYINILLFELPLNILIYNQRNHKSTTHHTLKIPITRLLCECELYAPSNYDNDPEMKEVMEIFDRQTSERFHEYDERMKTTRQKCKDKCDKEIQKIILKDKLEKELNEKFLTLQTDIQNDAIPTCVCEKSLADKVEKGCLRCGSILGAAMPEVGSIGGGLLYALNAWKPKALEAAIAAAKELAITEATNAGVKTVVSEINKLLAKFKQHEILFELKPIVNKSNFSCGSSLFQRAEELASKSCVAQPNGSYTSFCNTILNGEKTTFKPFAQAGANTYEKTLTTETPVLQARYTAAVKTAYGGYQTAIIASIVAIVVIVLIMVIIYLILRYRRKKKMKKKLQYIKLLEE

SEQ ID NO: 25 - protein, 329 aa, Plasmodium falciparum
MKVHYINILLFALPLNILVINQRNHNNSTYHTSNTKLTKTHRTLCECELYAPSNYENDPEMKELMENFNHQSSERFREYDERIQDKRKQFKEQCEKDIQKIILKDKIEKELTEKLSTLQTDISTNDIPTCVCEKSLADKMEKTCLKCGGVLGTAVPELGLIGGSVIYSAAQAAAAKLGVAKAIELMKKIYNLGNVSFIDWTNLINVGNYSHRMSLVGIVNKVNNMCQIKDPEGNVVFCFAKQNMRGGAGKFAQTISEQAGNAAIKAGETANVKFAEMTSVGTIFSDPIVISATVVVTIAVILIIIYLILRYRRKKKMKKKLQYIKLLEE

SEQ ID NO: 26 - protein, 371 aa, Plasmodium falciparum
MKIHYINILLFELPLNILIYNQRNYYITPRHTETNRSLCECELYSPTNYDNDPEMKRVMQQFVDRTTQRFHEYDERMKTTRQKCKERCDKEIQKIILKHKLEKELMDKFATLHTDIQSDAIPTCVCEKSLADKTEKFCHNCGYGLGSVAPNIGLLGGPGIYVWKIAALAAAKEFAEKAGAAMGKAAGDAAGAAELIRGLKALNIDKLFNESLGLVFDGTNYNNTEYIFKAIFSKFNESCMPRPPGSVPGPVIDRAFCDTVDTLVLPSGTGSQTSASTNAVIKEYVKPIVSNAKFTAEATAQTAAEEATNLALKTNTNAVNATYASSQTAIIVSIAAIVVIVLVMIIIYLILRYRRKKKMKKKLQYIKLLEE

SEQ ID NO: 27 - protein, 369 aa, Plasmodium falciparum
MKIHYTNILLFPLKLNILVNTHKKPSITARHIQTTRLLCECELFSPQNYDNDPEMKRVMQQFHDRTTQRFHEYDERMKTTRQECKEQCDKEIQKIILKDKMEKQMAEKLSTLETKINTDDIPTCVCEKSMADKTEKFCLNCGKNMAAIAPWWGLVCGSGYAGWLHSAMAAAIDKAIAEGAAAGIKAGHLAGTNAVIEQLRTLGIYFVGNKQLETIIDVTNYMNVSFIYDKVYSHYITLCTPRPVNGHLVSNFNFSDRFCKLFHQKDLVSLDIKSVKAIIKKNVEEAVAGAEQAAKAEVSNVTATKTTEFTTKNIAEVEAATTSYYTPIIASIVAIVIIVLIMVIIYKILRYRRKKKMKKKLQYIKLLEE

SEQ ID NO: 28 - protein, 369 aa, Plasmodium falciparum
MKVHYFNILLFALPLNILVSSPKKNPSITQKRPTRRLLCECELYAPANYDSVPQMKEVMDNFNRQTQQRFHEYDERMVEKRMQCKDKCDKEIQKIILKDKLEKQMKQELTTLETKITTDDIPTCICEKSLADKVEKGCLRCGGVFGGGVAPGVGLLGGIGQLGLDVWKAAAIKAATEYALTEGAAKGLAAGNAHGMNIVIYHLKELLIDKLVPNICKTVSSTGDYTRVINFSKLIIQKRGAMCGADGGTLSKDMCTQININLGTVLRNGKANLPDKEAVPKVLNRLVSQADKAANEVAKDTSQSVAVKITEQQTAAINATYTSWQIAITASVIAIVVIVLIMVIIYLILRYRRKKKMKKKLQYIKLLEE

SEQ ID NO: 29 - protein, 348 aa, Plasmodium falciparum
MKVHYINILLFALPLDILEHNKNEPHTTPNHTQTTRSLCECELYSPANNDNDPEMKRVMQQFEDRTSQRFHEYDERMVEKRMQCKDKCDKEIQKIILKDKLEKQMVEQFSTLQTDIQSDAIPTCVCEKSIEDKVEKGCLRCGSILGAAMPELGSVGGSLLYALNTWKPAAIIAAKEAALAEATDLATQAGIDTVVAQLKIEGLLASFTVKQRLVDLSSIVTSSTYNNGAILHKSAMELASSYCHFEGTQSTPPFCSTIKYGQTTNFVRYAKAGSAAFKTEFASKSATLTKAKVGAVEATYGGYHISIISSIVAIVVIVLIMVIIYLILRYRRKKKMKKKLQYIKLLEE

SEQ ID NO: 30 - protein, 377 aa, Plasmodium falciparum
MKVHYINILLFALPLNILIYNQRNHKSTTHHTLKIPTTRLLCECDLYIPNYDNDPQMKKIMENFDRQTSQRFHEYDERMKTTRQKCKDKCDKEIQKIILKDKIDKELTEKFATLQTDIQNDAIPTCVCEKSLADKVEKTCLKCGGVLGGGVTPAWGLISGIVYTGWKAAALAAAKKLAAEAGAAEGASQGAAAGATRLIELIQSTFQVQNIAGQSLESIFTAQTYTDVSNITKALFNEYAEICLPIFTDSVPVRGVRYNISSPICTFVEEGILATSRDKGGSPITFIEKKVETMVSKAEGVATARAADVAAAKTAEFEATKVGAVEATYAGYHTTIIASIIAILIIVLIMVIIYLILRYRRKKKMKKKLQYIKLLEE

SEQ ID NO: 31 - protein, 358 aa, Plasmodium falciparum
MKVHCYNILLFSFTLIILLLSSSQVNNQMNHYNTAHMKNTEPIKSYRSLCECELYTSMYDDDPEMKEILHDFDRQTSQRFEEYNERLLENKQKCKEQCEKDIQKIILKDKLEKELMDKFATLHTDIQSDAIPTCVCEKSIADKMEKECLRCAQNLGGIVAPSSGVLAGIAEGALIVWKPAAIKAAKAAAAKAASDAATQAGMNAVRLEIKKLLEMFTGKPGYVDLLPIVKESTYKNGSALVDSAKKLFVESGKLEGLDRMPVFYNTVIDYPGPSNIKGFGKIGSDAYEAAFTSQKGTLEATKVGEVNTTYGGCQTAITASVIAIVVIILIMVIIYLILRYRRKKKMKKKLQYIKLLEE

SEQ ID NO: 32 - protein, 367 aa, Plasmodium falciparum
MKDHYINILLFALPLNILVYNQRNYYITPRHTETNRSLCECELYSPTNYDSDPEMKRVMQQFVDRTTQRFHEYDESLQSKRKQCKDQCDKEIQKIILKDKIEKEFTEKLSTLQTDITTKDIPTCVCEKSLADKMEKVCLKCAQNLGGIVAPSTGVLGEIAALAVNAWKTTALKNAIAAAQKAGDAAGKIAGESKGVETIIGILEQYYSIYELKGTPLKSFFATTHYTDISNIATVIDTELNTSCGLNSLANQAICGLRTKLGLVAKPGQVMVTQKEAITKMITNVVHKSEITAEAAKTEVAATKTAAAIKMNTEAIEAATTPYYTPIIASIVAIVVIVLIMVIIYLILRYRRKKKMKKKLQYIKLLN

SEQ ID NO: 33 - protein, 378 aa, Plasmodium falciparum
MKFNYTNIILFSLSLNILLLSSRVYNKRNHKSIILHTSNENPIKTHRSLCECELYSPTNYDSDPEMKRVMQQFHDRTTQRFHEYDERMKTTRQECKEQCDKEIQKIILKDRLEKELMDKFATLHTDIQSDAIPTCVCEKSLADKTEKFCLNCGVQLGGGVLQASGLLGGIGQLGLDAWKAAALVTAKELAEKAGAAAGLKAGDIHGMKIVIEGLKALKVDTLKSGIFNSFVNNSHYTEVTGLAIAIDTEMNEVCSATYIGIHPICVVREKLGVIPKAGGTMVKQKDAITNVLKQALEKATQSAEALSETTAEDVAAKLTAQKTGAINTIFMSNQTAIIASIVAIVVIVLIMVIIYLILRYRRKKKMKKKLQYIKLLEE

SEQ ID NO: 34 - DNA, 2815 bp, Homo sapiens
ctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggcccccggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacacacagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctgcaggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggcagagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaaaaaaaaaaaaaaa

SEQ ID NO: 35 - protein, 652 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 36 - protein, 7 aa, Plasmodium falciparum
FHEYDER

SEQ ID NO: 37 - DNA, 25 bp, Artificial Sequence
  primer
ttatccttattttttaataactgcc

SEQ ID NO: 38 - DNA, 16 bp, Artificial Sequence
  primer
gttcgtggcattccac

SEQ ID NO: 39 - protein, 302 aa, Homo sapiens
MALLISLPGGTPAMAQILLLLSSACLHAGNSERSNRKNGFGVNQPESCSGVQGGSIDIPFSFYFPWKLAKDPQMSIAWRWKDFHGEFIYNSSLPFIHEHFKGRLILNWTQGQTSGVLRILNLKESDQTRYFGRVFLQTTEGIQFWQSIPGTQLNVTNATCTPTTLPSTTAATSAHTQNDITEVKSANIGGLDLQTTVGLATAAAVFLVGVLGLIVFLWWKRRRQGQKTKAEIPAREPLETSEKHESVGHEGQCMDPKENPKDNNIVYASISLSSPTSPGTAPNLPVHGNPQEETVYSIVKAK

SEQ ID NO: 40 - protein, 192 aa, Artificial Sequence
  Fushion protein of PILR alpha protein and conserved region of RIFIN protein 4
MRAWIFFLLCLAGRALAASDYKDDDDKLECECELYMSNYDNDPEMKRVMQQFHDRTTQRFHEYDDRMIEKRQKCKDRCNKEIEKIILKDKIEKELTETFATLNTNITNEDIPTCICKKSVADKIEKTCLKITVGLATAAAVFLVGVLGLIVFLWWKRRRQGQKTKAEIPAREPLETSEKHESVGHEGQCMDP

SEQ ID NO: 41 - protein, 201 aa, Artificial Sequence
  fushion protein of PILR alpha protein and variable region of RIFIN protein 4
MRAWIFFLLCLAGRALAASDYKDDDDKLEYETINAFIAKTIEELEGIPGITKLFGAKISQFVTPAVFRKPMSLVETILSEKKKLCLCAANKNELLCRGMNPNVPETLPKKIEVAVNEVLSSVNDTWATATTPTTFFTNPITVGLATAAAVFLVGVLGLIVFLWWKRRRQGQKTKAEIPAREPLETSEKHESVGHEGQCMDP